SEQUENCE LISTING (17 sequences)

SEQ ID NO: 1 - protein, 4 aa, Artificial Sequence
  EA-230
AQGV

SEQ ID NO: 2 - protein, 4 aa, Artificial Sequence
  Derivative of EA-230
AQLP

SEQ ID NO: 3 - protein, 4 aa, Artificial Sequence
  Derivative of EA-230
PLQA

SEQ ID NO: 4 - protein, 4 aa, Artificial Sequence
  Derivative of EA-230
LQGV

SEQ ID NO: 5 - protein, 4 aa, Artificial Sequence
  Derivative of EA-230
LAGV

SEQ ID NO: 6 - protein, 4 aa, Artificial Sequence
  Derivative of EA-230
PQVG

SEQ ID NO: 7 - protein, 4 aa, Artificial Sequence
  Derivative of EA-230
PQVA

SEQ ID NO: 8 - protein, 4 aa, Artificial Sequence
  Derivative of EA-230
VGQL

SEQ ID NO: 9 - protein, 4 aa, Artificial Sequence
  Derivative of EA-230
LQPL

SEQ ID NO: 10 - protein, 4 aa, Artificial Sequence
  Derivative of EA-230
LQVG

SEQ ID NO: 11 - protein, 4 aa, Artificial Sequence
  Derivative of EA-230
LQGA

SEQ ID NO: 12 - protein, 4 aa, Artificial Sequence
  Derivative of EA-230
AQGA

SEQ ID NO: 13 - protein, 4 aa, Artificial Sequence
  Derivative of EA-230
QPLA

SEQ ID NO: 14 - protein, 4 aa, Artificial Sequence
  Derivative of EA-230
PQVP

SEQ ID NO: 15 - protein, 4 aa, Artificial Sequence
  Derivative of EA-230
VGQA

SEQ ID NO: 16 - protein, 4 aa, Artificial Sequence
  Derivative of EA-230
QVGQ

SEQ ID NO: 17 - protein, 4 aa, Artificial Sequence
  Derivative of EA-230
VGQG

(19) United States
(12) Patent Application Publication
(10) Pub. No.: US 2022/0370543 A1
(43) Pub. Date: Nov. 24, 2022

(54) METHODS OF TREATMENT FOR MODIFYING HEMODYNAMICS

(71) Applicant: EBI ANTI SEPSIS B.V., 's-Gravenhage (NL)

(72) Inventors: Roelof Peter Pickkers, Nijmegen (NL); Gert Wensvoort, Koekange (NL)

(73) Assignees: EBI ANTI SEPSIS B.V., 's-Gravenhage (NL); EBI ANTI SEPSIS B.V., 's-Gravenhage (NL)

(21) Appl. No.: 17/765,162

(22) PCT Filed: Sep. 30, 2020
(86) PCT No.: PCT/NL2020/050605

§ 371 Date: Mar. 30, 2022

Related U.S. Application Data
(60) Provisional application No. 62/908,442, filed on Sep. 30, 2019.
(60) Provisional application No. 63/045,752, filed on Jun. 29, 2020.

(51) Int. Cl. A61K 38/07; A61P 7/04

(52) U.S. Cl. CPC A61K 38/07; A61P 7/04

(57) ABSTRACT

This disclosure provides method of treatment comprising administering an AQGV peptide, or a functional analog thereof, to a human subject, the human subject optionally having impaired kidney function, wherein the treatment of administering an AQGV peptide comprises maintaining or improving hemodynamic stability in the human subject, such as a human subject suffering or considered suffering from Clarkson's disease (CLS).

### CROSS-REFERENCE TO RELATED APPLICATIONS

This application is a national phase entry under 35 U.S.C. § 371 of International Patent Application PCT/NL2020/050605, filed Sep. 30, 2020, designating the United States of America and published as International Patent Publication WO 2021/066649 A1 on Apr. 8, 2021, which claims the benefit under Article 8 of the Patent Cooperation Treaty to U.S. Patent Application Ser. No. 62/908,442, filed Sep. 30, 2019, and to U.S. Patent Application Ser. No. 63/045,752, filed Jun. 29, 2020.

### TECHNICAL FIELD

This application relates generally to medicine, and more particularly to a method of treating a subject in need of maintaining or improving hemodynamic stability, a reduction in adverse vascular permeability, and/or a reduction in fluid retention. Such a method may include administering to the subject peptide(s) comprising at least 50% amino acids that are autophagy inhibiting amino acids, wherein the autophagy inhibiting amino acids are selected from the group consisting of alanine, glutamine, glycine, valine, leucine, isoleucine, proline, and arginine.

### BACKGROUND

When a human subject has been subjected to severe trauma, in particular trauma induced by medical interventions such as surgery, e.g., when having severe medical interventions such as open heart surgery, the human subject or patient, is admitted into an intensive care unit (ICU) where vital signs can be closely monitored. The patient receives medical treatment to allow the patient to recover and when vital signs are within acceptable boundaries, the patient can be released from ICU and admitted into standard hospital care. When the patient has shown to be stable at standard care, in particular when having shown sufficient hemodynamic stability, the patient can be released from the hospital and returns home. Subsequently, a patient can be readmitted into the hospital should the need arise because e.g., the condition of the patient worsens. Any improvement on the vital signs, i.e., the health and recovery of a patient affecting the length of stay of a patient in the ICU, the length of stay in standard care at the hospital and/or patient readmittance, provides for a significant benefit to patients and healthcare in general. Hence, any means and methods that improve the health and (rate of) recovery of a patient are of interest.

At the ICU or in the hospital, patients are often treated with fluid, e.g., with a salt containing aqueous solution, such as a physiological salt solution (e.g., 0.9% NaCl, also called saline) or any other suitable solution for infusion. Sometimes, when a patient is determined to be in need thereof, to such aqueous solution medication may be added. Usually, the aqueous solution containing medication is given intravenously (i.v.) by continuous (drip) infusion or by giving a bolus injection i.v. Other routes of fluid therapy may comprise intra-abdominal application by infusion or bolus of such fluid. Fluid balance (kidney function) is one of the determining criteria for patient outcome in ICU. For instance, hypervolemia is a medical condition when you have too much fluid in your body, also described as having excess water retention or fluid retention or commonly as water or fluid overload. Fluid therapy can result in fluid overload. Fluid overload can occur in human subjects, symptoms of which e.g., include weight gain and edema.

Fluid overload, and resulting inadequate blood flow or hemodynamic instability, often with a perceived need to administer fluid and/or vasopressor therapy, is a relatively frequent occurrence in critically ill patients and is often a consequence of critical care intervention with intravenous fluid therapy. Despite a common perception that it is benign, fluid overload in the critically ill is independently associated with increased morbidity and mortality. Fluid extravasation by capillary leakage into the interstitial space can adversely affect multiple organ systems, with various manifestations ranging from impaired cognition, impaired contractility of the heart, and tissue edema in skin and muscles giving rise to delayed wound healing, pressure ulcers and wound infection. In lungs, fluid overload induces increased extravascular lung water, with increased work of breathing and impaired gas exchanging leading to hypoxemia. A particular serious complication of fluid overload is kidney injury. It is known that fluid overload prolongs stay at the intensive care unit up to 60% and in the hospital up to 30%.

In general, fluid overload (FO) contributes to delayed recovery at ICU and prolonged length-of-stay at the hospital, and leads to increased health care resource uses and costs. A recent US-study (Child D et al., Clinicoecon Outcomes Res. 2015; 7:1-8) calculated total hospitalization cost per visit for a FO cohort at around fifteen thousand dollars higher than the non-FO cohort, which averaged out at over twenty-one thousand US dollars. The ICU cost for the FO cohort was over five thousand dollar higher than the non-FO cohort. FO patients had 16% higher mortality and 31% prolonged stay in the Hospital. More importantly this resulted in almost 60% longer stay in ICU and a significant increase in 30-day readmission, and ventilator usage over the non-FO cohort (all P<0.05). Diuretics are the most commonly used drugs to treat clinically diagnosed fluid overload. There is however no conclusive evidence that treatment with a diuretic alters major outcomes such as survival to hospital discharge or time in hospital.

The kidney is a highly vascular and encapsulated organ that is exquisitely sensitive to inadequate (insufficient or excess) blood flow. The kidney is particularly sensitive to venous congestion, and studies show that reduced venous return triggers a greater degree of kidney damage than that from lacking arterial flow. Intravenous fluid infusion, when exceeding the capacity of lymphatic drainage in the microcirculation, will inevitably cause interstitial edema. In the kidney, interstitial edema increases subcapsular and intra-capsular pressure, leading to the reduction in forward renal arterial blood flow, reduction in venous return and lymphatic drainage, ultimately causing tissue hypoxia and AKI. Inadequate urine output in AKI can further worsen tissue edema, creating a vicious cycle.

Such acute kidney injury is characterized by a rapid loss of renal function. Moreover, the acute injury often progresses into a chronic state, ultimately leading to end-stage renal disease. These patients are considered to be critically ill and require dialysis or renal replacement therapy. AKI exerts direct effects on other organs and systems as well and contributes to multi-organ failure in critically ill patients. AKI affects over 3 million patients per year with a mortality rate of up to 70% and is directly associated with short- and long-term complications in patients, and the condition is associated with a mortality rate of 40-70%. The mortality of patients with AKI is approximately 1 out of 4. Currently, the only treatment options for AKI are dialysis and supportive care, which do not address the underlying causes, do not limit further damage and do not prevent progression. Currently, no drugs are licensed to treat this condition.

Upon testing in a clinical trial aimed at assessing the safety and tolerability of an AQGV peptide (SEQ ID NO:1), also referred to as EA-230 herein, and its immunomodulatory effects, the peptide was found to be safe but, unexpectedly, no immunomodulatory effects were observed when comparing treated patients as compared with control subjects. While not observing immunomodulatory effects, it was found that, upon analysis of the data obtained in the clinical trial, new and highly advantageous properties could be attributed to the AQGV peptide that have not been observed before. These properties are apparently independent from known and observed immunomodulatory effects.

Surprisingly, in the absence of any observed immunomodulatory effects, it was found that the length of stay in the ICU (intensive care unit), and also the length of stay in hospital in general, of patients treated with the AQGV peptide, was significantly reduced. Upon an in depth analysis of parameters monitored in the human subjects during the study, it was found that the use of the AQGV peptide, advantageously modulated the hemodynamics of the treated patients. It was also found that parameters related to kidney function in human patients were shown to either have improved significantly, or were maintained at functional levels and did not deteriorate, even despite the absence of any observed immunomodulatory effects of treatment with AQGV peptide in these patients. Parameters related to kidney function and/or hemodynamics are generally monitored in patients and determine the length of stay in either ICU or hospital. The use of the AQGV peptide or functional equivalents thereof thus allows to advantageously improve parameters that are monitored in human patients to thereby reduce the length of stay in either ICU or hospital (see, for example, FIGS. 10, 15, 16).

### BRIEF SUMMARY

Hence, this disclosure relates to the use of a peptide herein also referred to as a hemodynamic peptide, and analogs (functional equivalents) thereof, for improving the clinical parameters of human patients admitted into hospital and/or intensive care such that the time period between admittance and release from hospital and/or intensive care can be shortened.

In one embodiment, disclosure provides a method of treatment of a human subject considered in need of maintaining or improving hemodynamic stability comprising administering to the subject a hemodynamic peptide wherein a hemodynamic peptide is defined as a peptide comprising at least 50%, more preferably at least 75%, most preferably 100% amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, the hemodynamic peptide comprises at least 50%, more preferably at least 75%, most preferably 100% amino acids selected from the group of autophagy inhibiting amino acids alanine (in one letter code: A), glutamine (Q), glycine (G), valine (V), leucine (L), and proline (P). In a more preferred embodiment, this disclosure provides a method of treatment of a human subject considered in need of maintaining or improving hemodynamic stability wherein the hemodynamic peptide comprises at least 50%, more preferably at least 75%, most preferably 100% amino acids selected from the group of autophagy inhibiting amino acids alanine (in one letter code: A), glutamine (Q), leucine (L), and proline (P). In another embodiment, this disclosure provides a method of treatment of a human subject considered in need of maintaining or improving hemodynamic stability wherein the peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that a hemodynamic peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO:1).

In another preferred embodiment, this disclosure provides a method of treatment of a human subject considered in need of maintaining or improving hemodynamic stability wherein the subject has been subjected to severe trauma such as surgery. In another embodiment, this disclosure provides a method of treatment of a human subject considered in need of maintaining or improving hemodynamic stability wherein the subject has been subjected to cancer treatment such as treatment with an antineoplastic (e.g., chemotherapy and/or radiotherapy) or immunomodulating agent. In another embodiment, this disclosure provides a method of treatment of a human subject considered in need of maintaining or improving hemodynamic stability wherein the subject is considered to suffer from capillary leakage syndrome such as seen with an adverse drug reaction. In another embodiment, this disclosure provides a method of treatment of a human subject considered in need of maintaining or improving hemodynamic stability the human subject having impaired organ function, in particular kidney function. In another embodiment, this disclosure provides a method of treatment of a human subject considered in need of maintaining or improving hemodynamic stability wherein the method comprises a reduced use of vasopressive agents. In another embodiment, this disclosure provides treatment of a human subject considered in need of maintaining or improving hemodynamic stability wherein the method comprises a reduced fluid intake. In another embodiment, this disclosure provides a method of treatment of a human subject considered in need of reducing adverse vascular permeability comprising administering to the subject hemodynamic peptide comprising at least 50% amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, the hemodynamic peptide comprises at least 50%, more preferably at least 75%, most preferably 100% amino acids selected from the group of autophagy inhibiting amino acids alanine (in one letter code: A), glutamine (Q), glycine (G), valine (V), leucine (L), and proline (P). In a more preferred embodiment, this disclosure provides a method of treatment of a human subject considered in need of reducing adverse vascular permeability wherein the hemodynamic peptide comprises at least 50%, more preferably at least 75%, most preferably 100% amino acids selected from the group of autophagy inhibiting amino acids alanine (in one letter code: A), glutamine (Q), leucine (L), and proline (P). In another embodiment, this disclosure provides a method of treatment of a human subject considered in need of reducing adverse vascular permeability wherein the peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that a hemodynamic peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO:1).

In another preferred embodiment, this disclosure provides a method of treatment of a human subject considered in need of reducing adverse vascular permeability wherein the subject has been subjected to severe trauma such as surgery comprising administering to the subject a hemodynamic peptide. In another embodiment, this disclosure provides a method of treatment of a human subject considered in need of reducing adverse vascular permeability wherein the subject has been subjected to cancer treatment such as treatment with an antineoplastic or immunomodulating agent. In another embodiment, this disclosure provides method of treatment of a human subject considered in need of reducing adverse vascular permeability wherein the subject is considered to suffer from capillary leakage syndrome such as seen with an adverse drug reaction. In another embodiment, this disclosure provides a method of treatment of a human subject considered in need of reducing adverse vascular permeability the human subject having impaired kidney function. In another embodiment, this disclosure provides a method of treatment of a human subject considered in need of reducing adverse vascular permeability wherein the method comprises a reduced use of vasopressive agents. In another embodiment, this disclosure provides a method of treatment of a human subject considered in need of reducing adverse vascular permeability wherein the method comprises a reduced fluid intake. In another embodiment, this disclosure provides a method of treatment of a human subject considered in need of reducing adverse fluid retention comprising administering to the subject a hemodynamic peptide comprising at least 50%, more preferably at least 75%, most preferably 100% % amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, the hemodynamic peptide comprises at least 50%, more preferably at least 75%, most preferably 100% amino acids selected from the group of autophagy inhibiting amino acids alanine (in one letter code: A), glutamine (Q), glycine (G), valine (V), leucine (L), and proline (P). In a more preferred embodiment, this disclosure provides a method of treatment of a human subject considered in need of reducing adverse fluid retention wherein the hemodynamic peptide comprises at least 50%, more preferably at least 75%, most preferably 100% amino acids selected from the group of autophagy inhibiting amino acids alanine (in one letter code: A), glutamine (Q), leucine (L), and proline (P). In another embodiment, this disclosure provides a method of treatment of a human subject considered in need of reducing adverse fluid retention wherein the peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the hemodynamic peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO:1).

In another preferred embodiment, this disclosure provides a method of treatment of a human subject considered in need of reducing adverse fluid retention wherein the subject has been subjected to severe trauma such as surgery. It is preferred that the hemodynamic peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO:1).

In another embodiment, this disclosure provides a method of treatment of a human subject considered in need of reducing adverse fluid retention wherein the subject has been subjected to cancer treatment such as treatment with an antineoplastic or immunomodulating agent. It is preferred that the hemodynamic peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO:1).

In another embodiment, this disclosure provides a method of treatment of a human subject considered in need of reducing adverse fluid retention wherein the subject is considered to suffer from capillary leakage syndrome such as seen with an adverse drug reaction. It is preferred that the hemodynamic peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO:1). In another embodiment, this disclosure provides a method of treatment of a human subject considered in need of reducing adverse fluid retention the human subject having impaired kidney function. It is preferred that the hemodynamic peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO:1). In another embodiment, this disclosure provides method of treatment of a human subject considered in need of reducing adverse fluid retention wherein the method comprises a reduced use of vasopressive agents. It is preferred that the hemodynamic peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV or for instance, a dimer or trimer, tetramer or pentamer thereof. In another embodiment, this disclosure provides a method of treatment of a human subject considered in need of reducing adverse fluid retention wherein the method comprises a reduced fluid intake. It is preferred that the AQGV peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide is AQGV (SEQ ID NO:1).

In another embodiment, this disclosure provides a method for use in treatment of a human subject considered in need of maintaining or improving hemodynamic stability wherein the hemodynamic peptide is a salt of an organic acid, preferably selected from the group of maleic acid, acetic acid, tartaric acid, citric acid. In another embodiment, this disclosure provides a method according for use in treatment of a human subject considered in need of maintaining or improving hemodynamic stability wherein the hemodynamic peptide is a salt of an organic acid, such as maleic acid, more preferably acetic acid, more preferably tartaric acid, most preferably citric acid.

In another embodiment, this disclosure provides a method for use in treatment of a human subject considered in need of reducing adverse vascular permeability wherein the hemodynamic peptide is a salt of an organic acid, preferably selected from the group of maleic acid, acetic acid, tartaric acid, citric acid. In another embodiment, this disclosure provides a method according for use in treatment of a human subject considered in need of reducing adverse vascular permeability wherein the hemodynamic peptide is a salt of an organic acid, such as maleic acid, more preferably acetic acid, more preferably tartaric acid, most preferably citric acid.

In another embodiment, this disclosure provides a method for use in treatment of a human subject considered in need of reducing adverse fluid retention wherein the hemodynamic peptide is a salt of an organic acid, preferably selected from the group of maleic acid, acetic acid, tartaric acid, citric acid. In another embodiment, this disclosure provides a method according for use in treatment of a human subject considered in need of reducing adverse fluid retention wherein the hemodynamic peptide is a salt of an organic acid, such as maleic acid, more preferably acetic acid, more preferably tartaric acid, most preferably citric acid.

In another embodiment, this disclosure provides a method for use in treatment of a human subject considered in need of maintaining or improving hemodynamic stability wherein the hemodynamic peptide is provided from a stock solution of a hemodynamic peptide-acetate, hemodynamic peptide tartrate or hemodynamic peptide citrate, preferably an aqueous solution, preferably wherein the stock solution is provided with or is prepared to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L of the of hemodynamic peptide-acetate, hemodynamic peptide tartrate or hemodynamic peptide citrate. In a more preferred embodiment, this disclosure provides a method for use in treatment of a human subject considered in need of maintaining or improving hemodynamic stability wherein the hemodynamic peptide is provided from a stock solution of the hemodynamic peptide-tartrate or the hemodynamic peptide-citrate wherein the concentration of the hemodynamic peptide is in the range of 2 mol/L to 2.5 mol/L. In a more preferred embodiment, this disclosure provides a method for use in treatment of a human subject considered in need of maintaining or improving hemodynamic stability wherein the hemodynamic peptide is provided from a stock solution of the hemodynamic peptide-citrate wherein the concentration of the peptide-citrate is in the range of 2.5 mol/L to 3 mol/L. In a more preferred embodiment, this disclosure provides a method for use in treatment of a human subject considered in need of maintaining or improving hemodynamic stability wherein the hemodynamic peptide is provided from a stock solution of the peptide-citrate wherein the concentration of the peptide-citrate is in the range of 3 mol/L to 3.5 mol/L. In a more preferred embodiment, this disclosure provides a method for use in treatment of a human subject considered in need of maintaining or improving hemodynamic stability wherein the hemodynamic peptide is provided from a stock solution of the peptide-citrate wherein the concentration of the peptide-citrate is in the range of 3.5 mol/L to 4.5 mol/L. In a more preferred embodiment, this disclosure provides a method for use in treatment of a human subject considered in need of maintaining or improving hemodynamic stability wherein the hemodynamic peptide is provided from a stock solution of the peptide-citrate wherein the concentration of the peptide-citrate is in the range of 4.5 mol/L to 5.5 mol/L. In a more preferred embodiment, this disclosure provides a method for use in treatment of a human subject considered in need of maintaining or improving hemodynamic stability wherein the hemodynamic peptide is provided from a stock solution of the peptide-citrate wherein the concentration of the peptide-citrate is equal to or larger than 5.5 mol/L. It is preferred that the stock solution is an aqueous solution.

In another embodiment, this disclosure provides a method for use in treatment of a human subject considered in need of reducing adverse vascular permeability wherein the hemodynamic peptide is provided from a stock solution of a hemodynamic peptide-acetate, hemodynamic peptide tartrate or hemodynamic peptide citrate, preferably an aqueous solution, preferably wherein the stock solution is provided with or is prepared to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L of the of hemodynamic peptide-acetate, hemodynamic peptide tartrate or hemodynamic peptide citrate. In a more preferred embodiment, this disclosure provides a method for use in treatment of a human subject considered in need of reducing adverse vascular permeability wherein the hemodynamic peptide is provided from a stock solution of the hemodynamic peptide-tartrate or the hemodynamic peptide-citrate wherein the concentration of the hemodynamic peptide is in the range of 2 mol/L to 2.5 mol/L. In a more preferred embodiment, this disclosure provides a method for use in treatment of a human subject considered in need of reducing adverse vascular permeability wherein the hemodynamic peptide is provided from a stock solution of the hemodynamic peptide-citrate wherein the concentration of the peptide-citrate is in the range of 2.5 mol/L to 3 mol/L. In a more preferred embodiment, this disclosure provides a method for use in treatment of a human subject considered in need of reducing adverse vascular permeability wherein the hemodynamic peptide is provided from a stock solution of the peptide-citrate wherein the concentration of the peptide-citrate is in the range of 3 mol/L to 3.5 mol/L. In a more preferred embodiment, this disclosure provides a method for use in treatment of a human subject considered in need of reducing adverse vascular permeability wherein the hemodynamic peptide is provided from a stock solution of the peptide-citrate wherein the concentration of the peptide-citrate is in the range of 3.5 mol/L to 4.5 mol/L. In a more preferred embodiment, this disclosure provides a method for use in treatment of a human subject considered in need of reducing adverse vascular permeability wherein the hemodynamic peptide is provided from a stock solution of the peptide-citrate wherein the concentration of the peptide-citrate is in the range of 4.5 mol/L to 5.5 mol/L. In a more preferred embodiment, this disclosure provides a method for use in treatment of a human subject considered in need of reducing adverse vascular permeability wherein the hemodynamic peptide is provided from a stock solution of the peptide-citrate wherein the concentration of the peptide-citrate is equal to or larger than 5.5 mol/L. It is preferred that the stock solution is an aqueous solution.

In another embodiment, this disclosure provides a method for use in treatment of a human subject considered in need of reducing adverse fluid retention wherein the hemodynamic peptide is provided from a stock solution of a hemodynamic peptide-acetate, hemodynamic peptide tartrate or hemodynamic peptide citrate, preferably an aqueous solution, preferably wherein the stock solution is provided with or is prepared to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L of the of hemodynamic peptide-acetate, hemodynamic peptide tartrate or hemodynamic peptide citrate. In a more preferred embodiment, this disclosure provides a method for use in treatment of a human subject considered in need of reducing adverse fluid retention wherein the hemodynamic peptide is provided from a stock solution of the hemodynamic peptide-tartrate or the hemodynamic peptide-citrate wherein the concentration of the hemodynamic peptide is in the range of 2 mol/L to 2.5 mol/L. In a more preferred embodiment, this disclosure provides a method for use in treatment of a human subject considered in need of reducing adverse fluid retention wherein the hemodynamic peptide is provided from a stock solution of the hemodynamic peptide-citrate wherein the concentration of the peptide-citrate is in the range of 2.5 mol/L to 3 mol/L. In a more preferred embodiment, this disclosure provides a method for use in treatment of a human subject considered in need of reducing adverse fluid retention wherein the hemodynamic peptide is provided from a stock solution of the peptide-citrate wherein the concentration of the peptide-citrate is in the range of 3 mol/L to 3.5 mol/L. In a more preferred embodiment, this disclosure provides a method for use in treatment of a human subject considered in need of reducing adverse fluid retention wherein the hemodynamic peptide is provided from a stock solution of the peptide-citrate wherein the concentration of the peptide-citrate is in the range of 3.5 mol/L to 4.5 mol/L. In a more preferred embodiment, this disclosure provides a method for use in treatment of a human subject considered in need of reducing adverse fluid retention wherein the hemodynamic peptide is provided from a stock solution of the peptide-citrate wherein the concentration of the peptide-citrate is in the range of 4.5 mol/L to 5.5 mol/L. In a more preferred embodiment, this disclosure provides a method for use in treatment of a human subject considered in need of reducing adverse fluid retention wherein the hemodynamic peptide is provided from a stock solution of the peptide-citrate wherein the concentration of the peptide-citrate is equal to or larger than 5.5 mol/L. It is preferred that the stock solution is an aqueous solution.

In another embodiment, this disclosure provides a method for use in treatment of a human subject suffering or considered suffering from Clarkson's disease (CLS) wherein the hemodynamic peptide is provided from a stock solution of a hemodynamic peptide-acetate, hemodynamic peptide tartrate or hemodynamic peptide citrate, preferably an aqueous solution, preferably wherein the stock solution is provided with or is prepared to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L of the of hemodynamic peptide-acetate, hemodynamic peptide tartrate or hemodynamic peptide citrate. In a more preferred embodiment, this disclosure provides a method for use in treatment of a human subject suffering or considered suffering from Clarkson's disease (CLS) wherein the hemodynamic peptide is provided from a stock solution of the hemodynamic peptide-tartrate or the hemodynamic peptide-citrate wherein the concentration of the hemodynamic peptide is in the range of 2 mol/L to 2.5 mol/L. In a more preferred embodiment, this disclosure provides a method for use in treatment of a human subject suffering or considered suffering from Clarkson's disease (CLS) wherein the hemodynamic peptide is provided from a stock solution of the hemodynamic peptide-citrate wherein the concentration of the peptide-citrate is in the range of 2.5 mol/L to 3 mol/L. In a more preferred embodiment, this disclosure provides a method for use in treatment of a human subject suffering or considered suffering from Clarkson's disease (CLS) wherein the hemodynamic peptide is provided from a stock solution of the peptide-citrate wherein the concentration of the peptide-citrate is in the range of 3 mol/L to 3.5 mol/L. In a more preferred embodiment, this disclosure provides a method for use in treatment of a human subject considered suffering or considered suffering from Clarkson's disease (CLS) wherein the hemodynamic peptide is provided from a stock solution of the peptide-citrate wherein the concentration of the peptide-citrate is in the range of 3.5 mol/L to 4.5 mol/L. In a more preferred embodiment, this disclosure provides a method for use in treatment of a human subject suffering or considered suffering from Clarkson's disease (CLS) wherein the hemodynamic peptide is provided from a stock solution of the peptide-citrate wherein the concentration of the peptide-citrate is in the range of 4.5 mol/L to 5.5 mol/L. In a more preferred embodiment, this disclosure provides a method for use in treatment of a human subject suffering or considered suffering from Clarkson's disease (CLS) wherein the hemodynamic peptide is provided from a stock solution of the peptide-citrate wherein the concentration of the peptide-citrate is equal to or larger than 5.5 mol/L. It is preferred that the stock solution is an aqueous solution.

In another embodiment, this disclosure provides a hemodynamic peptide for use in treatment of a human subject considered in need of maintaining or improving hemodynamic stability the peptide comprising at least 50%, more preferably at least 75%, most preferably 100% amino acids selected from the group of autophagy inhibiting amino acids alanine (in one letter code: A), glutamine (Q), glycine (G), valine (V), leucine (L), isoleucine (I), proline (P) and arginine (R), the hemodynamic peptide more preferably comprises at least 50%, more preferably at least 75%, most preferably 100% amino acids selected from the group of autophagy inhibiting amino acids alanine (in one letter code: A), glutamine (Q), glycine (G), valine (V), leucine (L), and proline (P), preferably the hemodynamic peptide comprises at least 50%, more preferably at least 75%, more preferably 100% amino acids selected from the group of autophagy inhibiting amino acids alanine (in one letter code: A), glutamine (Q), leucine (L), and proline (P) or the peptide comprises at least 50%, more preferably at least 75%, most preferably 100% amino acids selected from the group of autophagy inhibiting amino acids alanine (in one letter code: A), glutamine (Q), glycine (G) and valine (V). Most preferably, the peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide for use in treatment of a human subject considered in need of maintaining or improving hemodynamic stability is AQGV (SEQ ID NO:1).

In another embodiment, this disclosure provides a hemodynamic peptide for use in treatment of a human subject considered in need of reducing adverse vascular permeability the peptide comprising at least 50%, more preferably at least 75%, most preferably 100% amino acids selected from the group of autophagy inhibiting amino acids alanine (in one letter code: A), glutamine (Q), glycine (G), valine (V), leucine (L), isoleucine (I), proline (P) and arginine (R), the hemodynamic peptide more preferably comprises at least 50%, more preferably at least 75%, most preferably 100% amino acids selected from the group of autophagy inhibiting amino acids alanine (in one letter code: A), glutamine (Q), glycine (G), valine (V), leucine (L), and proline (P), preferably the hemodynamic peptide comprises at least 50%, more preferably at least 75%, more preferably 100% amino acids selected from the group of autophagy inhibiting amino acids alanine (in one letter code: A), glutamine (Q), leucine (L), and proline (P) or the peptide comprises at least 50%, more preferably at least 75%, most preferably 100% amino acids selected from the group of autophagy inhibiting amino acids alanine (in one letter code: A), glutamine (Q), glycine (G) and valine (V). Most preferably, the peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide for use in treatment of a human subject considered in need of maintaining or improving hemodynamic stability is AQGV (SEQ ID NO:1).

In another embodiment, this disclosure provides a hemodynamic peptide treatment of a human subject considered in need of reducing adverse fluid retention, the peptide comprising at least 50%, more preferably at least 75%, most preferably 100% amino acids selected from the group of autophagy inhibiting amino acids alanine (in one letter code: A), glutamine (Q), glycine (G), valine (V), leucine (L), isoleucine (I), proline (P) and arginine (R), the hemodynamic peptide more preferably comprises at least 50%, more preferably at least 75%, most preferably 100% amino acids selected from the group of autophagy inhibiting amino acids alanine (in one letter code: A), glutamine (Q), glycine (G), valine (V), leucine (L), and proline (P), preferably the hemodynamic peptide comprises at least 50%, more preferably at least 75%, more preferably 100% amino acids selected from the group of autophagy inhibiting amino acids alanine (in one letter code: A), glutamine (Q), leucine (L), and proline (P) or the peptide comprises at least 50%, more preferably at least 75%, most preferably 100% amino acids selected from the group of autophagy inhibiting amino acids alanine (in one letter code: A), glutamine (Q), glycine (G) and valine (V). Most preferably, the peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide for use in treatment of a human subject considered in need of reducing adverse fluid retention is AQGV (SEQ ID NO:1).

In another embodiment, this disclosure provides a hemodynamic peptide treatment of a human subject considered in need of reducing adverse fluid retention, the peptide comprising at least 50%, more preferably at least 75%, most preferably 100% amino acids selected from the group of autophagy inhibiting amino acids alanine (in one letter code: A), glutamine (Q), glycine (G), valine (V), leucine (L), isoleucine (I), proline (P) and arginine (R), the hemodynamic peptide more preferably comprises at least 50%, more preferably at least 75%, most preferably 100% amino acids selected from the group of autophagy inhibiting amino acids alanine (in one letter code: A), glutamine (Q), glycine (G), valine (V), leucine (L), and proline (P), preferably the hemodynamic peptide comprises at least 50%, more preferably at least 75%, more preferably 100% amino acids selected from the group of autophagy inhibiting amino acids alanine (in one letter code: A), glutamine (Q), leucine (L), and proline (P) or the peptide comprises at least 50%, more preferably at least 75%, most preferably 100% amino acids selected from the group of autophagy inhibiting amino acids alanine (in one letter code: A), glutamine (Q), glycine (G) and valine (V). Most preferably, the peptide comprises at least 50%, more preferably at least 75%, most preferably 100% amino acids selected from the group of autophagy inhibiting amino acids alanine (in one letter code: A), glutamine (Q), glycine (G) and valine (V). It is preferred that the peptide for use in treatment of a human subject considered in need of reducing adverse fluid retention is AQGV (SEQ ID NO:1).

In another embodiment, this disclosure provides a hemodynamic peptide according to this disclosure wherein the subject has been subjected to severe trauma such as surgery.

In another embodiment, this disclosure provides a peptide according to this disclosure wherein the subject has been subjected to cancer treatment such as treatment with an antineoplastic or immunomodulating agent. In another embodiment, this disclosure provides a hemodynamic peptide according to this disclosure wherein the subject is considered to suffer from capillary leakage syndrome such as seen with an adverse drug reaction.

In another embodiment, this disclosure provides a hemodynamic peptide according to this disclosure, the human subject having impaired kidney function. In another embodiment, this disclosure provides a hemodynamic peptide according to this disclosure, wherein the use comprises (results in) a reduced use of vasopressive agents. In another embodiment, this disclosure provides a hemodynamic peptide according to this disclosure, wherein the use comprises (results in) a reduced fluid intake. In another embodiment, this disclosure provides a hemodynamic peptide according to this disclosure wherein the peptide is a salt of an organic acid, preferably selected from the group of maleic acid, acetic acid, tartaric acid, and citric acid. In another embodiment, this disclosure provides a hemodynamic peptide according to this disclosure wherein the hemodynamic peptide is a salt of an organic acid, such as maleic acid, more preferably acetic acid, more preferably tartaric acid, most preferably citric acid. In another embodiment, this disclosure provides a method wherein the hemodynamic peptide is provided from a stock solution of a hemodynamic peptide wherein the hemodynamic peptide is a hemodynamic peptide-acetate, hemodynamic peptide tartrate or hemodynamic peptide citrate, preferably an aqueous solution, preferably wherein the stock solution is provided with or is prepared to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L of the of hemodynamic peptide-acetate, hemodynamic peptide tartrate or hemodynamic peptide citrate. In a more preferred embodiment, this disclosure provides a stock-solution of the hemodynamic peptide-tartrate or the hemodynamic peptide-citrate wherein the concentration of the hemodynamic peptide is in the range of 2 mol/L to 2.5 mol/L. In a more preferred embodiment, this disclosure provides a stock-solution of the hemodynamic peptide-citrate wherein the concentration of the peptide-citrate is in the range of 2.5 mol/L to 3 mol/L. In a more preferred embodiment, this disclosure provides a stock-solution of the peptide-citrate wherein the concentration of the peptide-citrate is in the range of 3 mol/L to 3.5 mol/L. In a more preferred embodiment, this disclosure provides a stock-solution of the peptide-citrate wherein the concentration of the peptide-citrate is in the range of 3.5 mol/L to 4.5 mol/L. In a more preferred embodiment, this disclosure provides a stock-solution of the peptide-citrate wherein the concentration of the peptide-citrate is in the range of 4.5 mol/L to 5.5 mol/L. In a more preferred embodiment, this disclosure provides a stock-solution of the peptide-citrate wherein the concentration of the peptide-citrate is equal to or larger than 5.5 mol/L. It is preferred that the stock solution is an aqueous solution.

In another embodiment, this disclosure provides a pharmaceutical formulation comprising a hemodynamic peptide according to this disclosure. In another embodiment, this disclosure provides a pharmaceutical formulation according to this disclosure and at least one pharmaceutically acceptable excipient. It is preferred that the formulation is a stock-solution of hemodynamic-peptide. Preferably, a stock solution of a hemodynamic peptide-acetate, hemodynamic peptide tartrate or hemodynamic peptide citrate, preferably an aqueous solution, preferably wherein the stock solution is provided with or is prepared to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L of the hemodynamic peptide-acetate, hemodynamic peptide tartrate or hemodynamic peptide citrate. In a more preferred embodiment, this disclosure provides a pharmaceutical formulation comprising a stock solution of the hemodynamic peptide-tartrate or the hemodynamic peptide-citrate wherein the concentration of the hemodynamic peptide is in the range of 2 mol/L to 2.5 mol/L. In a more preferred embodiment, this disclosure provides a pharmaceutical formulation comprising a stock solution of the hemodynamic peptide-citrate wherein the concentration of the peptide-citrate is in the range of 2.5 mol/L to 3 mol/L. In a more preferred embodiment, this disclosure provides a method for use in treatment of a human subject considered in need of maintaining or improving hemodynamic stability wherein the hemodynamic peptide is provided from a stock solution of the peptide-citrate wherein the concentration of the peptide-citrate is in the range of 3 mol/L to 3.5 mol/L. In a more preferred embodiment, this disclosure provides a pharmaceutical formulation comprising a stock solution of the hemodynamic peptide-citrate wherein the concentration of the peptide-citrate is in the range of 3.5 mol/L to 4.5 mol/L. In a more preferred embodiment, this disclosure provides a pharmaceutical formulation comprising a stock solution of the hemodynamic peptide-citrate wherein the concentration of the peptide-citrate is in the range of 4.5 mol/L to 5.5 mol/L. In a more preferred embodiment, this disclosure provides a pharmaceutical formulation comprising a stock solution of the hemodynamic peptide-citrate wherein the concentration of the peptide-citrate is equal to or larger than 5.5 mol/L. It is preferred that the stock solution is an aqueous solution.

In one embodiment, the use of a hemodynamic peptide, and analogs thereof, is for use in a medical treatment for modifying hemodynamics in human subjects, in particular in subjects with resulting impaired kidney function. In a further embodiment, the use in human subjects for modifying hemodynamics, involves a reduction of reducing undesired fluid retention (i.e., undesired fluid overload) and/or a reduced use of vasopressive/inotropic agents in the human subject, in particular in subjects with resulting impaired kidney function. In another embodiment, the use of a hemodynamic peptide, and analogs thereof, is for use in human subjects having capillary leakage, in particular in subjects with resulting impaired kidney function.

In one embodiment, a hemodynamic peptide, or a functional analog thereof, is provided for use in the treatment of a human subject, the use comprises a treatment for modifying hemodynamics in the human subject. Hemodynamics involves the dynamics of blood flow, i.e., the physical factors that govern blood flow through the human body. Hemodynamics in human patients can be monitored by measuring e.g., blood pressure and/or the fluid balance. When blood pressure is low and/or the fluid balance disturbed in a human patient, vasopressors, or inotropes may be used and/or fluid administered, e.g., intravenously. Inotropes and vasopressors are biologically and clinically important vasoactive medications that originate from different pharmacological groups and act at some of the most fundamental receptor and signal transduction systems in the body. More than 20 such agents are in common clinical use, yet few reviews of their pharmacology exist outside of physiology and pharmacology textbooks. Despite widespread use in critically ill patients, understanding of the clinical effects of these drugs in pathological states is poor. Adverse effects of vasopressors and inotropes depend on the mechanism of action. For the medications that have beta stimulation, arrhythmias are one of the most common adverse effects that one would like to reduce.

It has been found that by using a hemodynamic peptide, or a functional analog thereof, the hemodynamics in human patients post-trauma were significantly improved as shown by e.g., a reduced use of vasopressors and/or an improved fluid balance in human patients. The use of a hemodynamic peptide, or a functional analog (as defined herein) thereof, as described herein thus improves the hemodynamic stability in human patients. Modifying or optimizing hemodynamics in human subjects is of importance post-surgery or post-injury, when e.g., human subjects have suffered trauma and/or blood loss. Hence, the hemodynamic peptide, or an analog thereof, can advantageously be used in hemodynamic therapy. Hemodynamic therapy, i.e., the optimization of hemodynamics in patients, includes perioperative hemodynamic therapy and/or goal-directed hemodynamic therapy. Such therapies can include therapeutic interventions such as fluid management in patients and/or the use of vasopressors.

AQGV (SEQ ID NO:1) functional analogs are defined herein as peptides exerting analogous effect or function as the hemodynamic peptide as described herein, in kind not necessarily in amount. They may be used according to this disclosure as single species peptides, in combination with other analogs and/or hemodynamic peptides in any desired ratio to modulate half live of the resulting mixture. An AQGV (SEQ ID NO:1) functional analog may have sequence identity, i.e., comprising at least part or the whole of the hemodynamic peptide. Preferably, such a hemodynamic functional analog is a structural analog of the hemodynamic peptide. A preferred structural analog may be an LQGV peptide. Structural analogs of the hemodynamic peptide may be selected from peptides comprising amino acids selected from the group of amino acids alanine (in one letter code: A), glutamine (Q), glycine (G), valine (V), leucine (L), and proline (P). A, Q, G, V and L are preferred. A, Q, G and V are most preferred in any order and ratio to one another and in a length of 4-30 amino acids, preferably 4-12 amino acids. In a preferred embodiment, this disclosure provides for a AQGV (SEQ ID NO: 1) structural analog, which comprises at least 50%, more preferably at least 75%, most preferably at least 100% amino acids selected from the group of autophagy inhibiting amino acids alanine (in one letter code: A), glutamine (Q), glycine (G), valine (V), leucine (L), proline (P), and arginine (R). The ratios between the amino acids may vary, but the peptide must comprise at least three different amino acids and Q should be present. Preferably a structural analog of the hemodynamic peptide has a length in the range of 4-30, more preferably 4-12 amino acids. Preferably, such a structural analog is a linear peptide. Suitable structural analogs of hemodynamic may have a length less than 4, e.g., of 3, however such lengths may require higher doses of such peptides because the half-life of such peptides will be shorter and thus less preferred. Longer structural analogs, e.g., longer than 30 residues, are less preferred because of potential immunogenicity of such longer peptides. A structural hemodynamic analog according to this disclosure may be selected from the group of peptides comprising a tetrapeptide selected from the group of AQLP, PLQA, LQGV, LAGV, PQVG, PQVA, VGQL, LQPL, LQVG, LQGA, AQGA, QPLA, PQVP, VGQA, QVGQ, and VGQG.

Vasopressors are a class of drugs that can elevate low blood pressure. Some vasopressors act as vasoconstrictors, other vasopressor sensitize adrenoreceptors to catecholamines—glucocorticoids, and another class of vasopressors can increase cardiac output. Whichever vasopressor is used, this disclosure allows for a reduction in the use of vasopressors. A reduction in the use of vasopressors involves a reduction in the amount of vasopressors used, i.e., the duration of vasopressor use is reduced and/or the dosage of the vasopressor is reduced. Examples of vasopressors are e.g., epinephrine, noradrenaline, phenylephrine, dobutamine, dopamine, and vasopressin. Fluid management in patients involves monitoring e.g., oral, enteral, and/or intravenous intake of fluids and fluid output (e.g., urine) and subsequently managing fluid intake e.g., in case of an observed fluid retention (i.e., the fluid intake exceeds fluid output, there is an overload situation). Strikingly, the use of the hemodynamic peptide, or an analog thereof, can reduce fluid retention, herein also called fluid overload. Hence, the hemodynamic peptide, or functional analog thereof, can be used in addition to known interventions that are to improve the hemodynamics in human patients, thereby resulting in a faster improvement in hemodynamics as compared with not using a hemodynamic peptide, or an analog thereof.

In another embodiment, a hemodynamic peptide, or a functional analog thereof, is provided for use in the treatment of a human subject having impaired kidney function. In a further embodiment, the impaired kidney function is acute kidney injury (AKI). In one embodiment, a hemodynamic peptide, or a functional analog thereof, is provided for use in the treatment of a human subject for improving kidney function. Kidney function can be assessed by determining the glomerular filtration rate (GFR), for example by assessing the clearance of iohexol from blood plasma. Kidney function can also be assessed by measuring plasma levels of creatinine and calculating an estimated GFR (eGFR) function therefrom, also referred to as the MDRD (Modification of Diet in Renal Disease) formula or equation, taking into account patient characteristics such as sex, age and race. Kidney function can be assessed based on GFR measurements (or estimates thereof based on MDRD) by applying the RIFLE criteria (see FIG. 3). Having a RIFLE score that is in the stage of risk, injury, failure, loss or ESKD, can be indicative of kidney injury and/or impairment of kidney function. Assessing kidney function in humans is standard clinical practice (e.g., by determining GFR, creatinine clearance, and/or eGFR/MDRD). Improvements in kidney function as compared with not receiving the hemodynamic peptide can include progressing to a kidney function stage as assessed under the RIFLE criteria to a less severe stage (e.g., a patient progressing from having injury to being at risk of injury or having no AKI). Improvements in kidney function also include having an improvement in GFR or eGFR scores. Irrespective of what assessment is made, the use of the hemodynamic peptide, or analog thereof, can improve kidney function in humans having kidney injury and/or an impairment of kidney function in subjects absent of immunomodulatory effects.

The use of the hemodynamic peptide allows for improving kidney function but it can also prevent a reduction and/or an impairment of kidney function (for examples, see FIGS. 6, 7 and 8). Accordingly, AKI may be prevented. Preferably, in prevention of a human subject having impaired kidney function, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, though the administration may also be longer such as upwards of 3 hours. The actual administration time may be determined by the physician. Hence, in one embodiment, the use of the hemodynamic peptide, or functional analog thereof, allows maintenance of kidney function in human patients. Hence, the use of the hemodynamic peptide, or analog thereof allows for the protection of kidney function in human patients. In another embodiment, the use of the hemodynamic peptide, or analog thereof, prevents a reduction and/or impairment of kidney function in human patients. For example, a human patient that may be classified as having no AKI, or being at risk of having kidney injury (such as AKI), when such a patient receives treatment with the hemodynamic peptide, such a patient may maintain its status instead of progressing to a lack of kidney function at a more severe stage. Hence, human patients that are at risk of developing kidney injury, e.g., due to (induced) trauma, such human patients as a result of receiving treatment with the hemodynamic peptide, or analog thereof, can maintain their kidney function status.

In another embodiment, a hemodynamic peptide, or a functional analog thereof, is provided for use in the treatment of a human subject having impaired kidney function and the use comprises modifying hemodynamics in the human subject. As treatment of kidney function and treatment of hemodynamic stability can now be linked, the use of a hemodynamic peptide, or a functional analog thereof, in accordance with this disclosure can advantageously be used to protect kidney function and/or improve kidney function, and modifying hemodynamics. Such combined use resulting e.g., in improved and/or maintained kidney function and a reduction in the use of vasopressors and/or improved fluid management in human subjects (for examples, see Tables 1, 3, 4 and FIGS. 14, 18, 19 and 20). Preferably, in the treatment of a human subject having impaired kidney function, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours, more preferably at least 3.5 hours, more preferably at least 4.5 hours.

In a further embodiment, this disclosure provides for a reduced use of vasopressive agents. It is understood that a reduced use of vasopressive agents can comprise reducing the amount of vasopressive agents used. The use of vasopressive agents can be reduced by reducing the duration of the use of vasopressive agents. The use of vasopressive agents can be reduced by reducing the amount of vasopressive agents (e.g., reducing amount per dosage and/or increasing time interval between administrations). The use of vasopressive agents can be reduced by reducing the amount of vasopressive agents and the duration of the use of vasopressive agents. By reducing the use of vasopressive agents, human subjects advantageously recover more quickly as compared with human subjects not receiving the hemodynamic peptide, or an analog thereof. Preferably, in use in reducing the amount and/or duration of use of vasopressive agents, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours or more. In another embodiment, the use of a hemodynamic peptide, or a functional analog thereof, reduces adverse fluid retention in the human subject. Leakiness of capillaries, fluid retention or fluid overload can occur in human subjects, symptoms of which e.g., include weight gain and edema. Fluid retention, otherwise known as swelling or edema, or capillary leakage, is a build-up of fluid in the body. As the fluid leaks out from the bloodstream, blood volume and blood pressure may drop. This can starve tissues in the kidneys, brain and liver of the oxygen and nutrients these organs need for normal function. Such swelling most often affects the dependent extremities (like the feet, ankles and hands) but swelling can also affect other parts of the body, such as organ cavities or the abdomen, or brains. Causes of swelling may be related to medication, heart disease, liver disease, or kidney failure. Cancer treatments, such as radiation therapy or some chemotherapy drugs can cause fluid retention in the body. This form of cancer swelling is most noticeable in the feet, ankles, hands, and face. It is a vascular reaction that causes an increased ability for fluid in the capillaries to “leak” into the layers of the skin, resulting in swelling. This happens much less often than hives alone. The fluid retention causes swelling generally in the tongue, lips, or eyelids. Swelling of the airways can result in difficult breathing, closing off of the airway and in the worst case death. Swelling of brains is often associated with—or follows—a dysfunction of the blood-brain barrier and edema formation after neurotrauma. Such traumatic brain injury (TBI) is the leading cause of death and long-term disability in developed countries, particularly affecting the young population and elderly. One of the major clinical problems associated with TBI, as well as other types of brain injury, such as subarachnoid or intracerebral hemorrhage and ischemic stroke, is the formation of cerebral edema—a rapid swelling of neural tissue, which, when uncontrolled, may result in death.

Another concern is capillary leak syndrome (CLS, also called systemic capillary leak syndrome (SCLS) or Clarkson's disease (CLS)), a disorder characterized by repeated flares of massive leakage of plasma from blood vessels into neighboring body cavities and muscles. This may result in a sharp drop in blood pressure that, if not treated, can lead to organ failure and death. Capillary leak syndrome (CLS) is a rare disease with profound vascular leakage, which is associated with a high mortality. The disease can also occur in cancer patients and effective therapeutic strategies have not been established yet. CLS can be idiopathic or secondary to autoimmune diseases, malignant hematological diseases, snakebites, and treatments such as chemotherapies and therapeutic growth factors. It lately becomes more and more apparent that the drugs adversely associated with CLS are commonly used in practice. There have been several reports on CLS as an adverse effect of anti-cancer agents and therapy, and the incidence of CLS, according to the kinds of anti-cancer drugs, has been systemically evaluated (PMID: 30691103). The largest number of studies reported on CLS incidence during interleukin-2 (IL-2) treatment, the second largest number of studies reported on anti-cluster of differentiation (anti-CD) agents. Also, use of an antineoplastic or immunomodulating agent, such as anti-cancer agents and anti-cancer immunotherapy, including IL-2+imatinib, mesylate and monoclonal antibodies (mAb) such as rituximab, showed a dose-dependent increase in the incidence of CLS as an adverse event of anti-cancer treatment. Similarly, Clarkson's disease (CLS) is commonly reported as (a suspected) adverse drug reaction (ADR) in human clinical trials as a fairly common adverse reaction to (experimental or investigational) drug testing.

Preferably, in treatment of a human subject having fluid retention, the hemodynamic peptide as defined herein is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours, more preferably at least 3.5 hours, more preferably at least 4.5 hours. Hence, in one embodiment, the use of the hemodynamic peptide, or analog thereof, allows to treat a fluid retention in human patients. Hence, the use of the hemodynamic peptide, or analog thereof protects against fluid retention in human patients. In a preferred embodiment, the use of the hemodynamic peptide, or analog thereof, protects against fluid retention, such as Clarkson's disease (CLS), in a human patient receiving anti-cancer-treatment, such as treatment with an antineoplastic or immunomodulating agent.

In another embodiment, the use of the hemodynamic peptide, or analog thereof, prevents fluid retention in human patients. In prevention of fluid retention in a human subject, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, or at least 2.5 hours or more. In a preferred embodiment, the use of the hemodynamic peptide, or analog thereof, prevents fluid retention, such as Clarkson's disease (CLS), in a human patient receiving anti-cancer-treatment, such as treatment with an antineoplastic or immunomodulating agent affecting capillary leakage. In another preferred embodiment, the use of the hemodynamic peptide, or analog thereof, prevents fluid retention, such as Clarkson's disease (CLS), in a human patient having an adverse drug reaction, such as after (experimental) treatment with an, often antineoplastic or immunomodulating, drug affecting capillary leakage.

Fluid retention can be the result of reduced kidney function and/or impaired hemodynamics. Hence, because the use of hemodynamic peptide can affect kidney function and/or hemodynamics in human subjects, the use of hemodynamic peptide can affect fluid retention as well. Fluid retention can be the result of leaky capillaries. Hence, the use of hemodynamic peptide, and/or analogs thereof, may have an effect on the leakiness of capillaries, reducing leakage of plasma from the blood to peripheral tissue and/or organs. Most preferably edema can be reduced and/or avoided by the use of hemodynamic peptide. Such may also be referred to as adverse fluid retention as it has an adverse effect on the patient. Whichever is the cause of fluid retention, the use of a hemodynamic peptide, and/or a functional analog thereof can improve fluid retention in human subjects thereby alleviating symptoms associated with fluid retention such as weight gain and edema, which subsequently can reduce the use of diuretics. Preferably, in use of hemodynamic peptide to improve fluid retention, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours, more preferably at least 3.5 hours, more preferably at least 4.5 hours.

In another embodiment, the use of the hemodynamic peptide, or a functional analog thereof, in accordance with this disclosure, is not restricted to patients having kidney injury and/or requiring hemodynamic therapy. The use of a hemodynamic peptide, and/or a functional analog thereof, in accordance with this disclosure, includes the treatment of human patients that are believed to be at risk of having kidney injury and/or anticipated to require hemodynamic therapy. Such human patients include patients that are to be admitted, or are expected to be admitted, into intensive care. Hence, the use of the hemodynamic peptide, or a functional analog thereof, includes a use for induced trauma, such as surgery, as shown e.g., in the examples. Induced trauma includes any physical injury to the human body and typically can include the loss of blood and/or injury to tissues of the human subject. Induced trauma includes e.g., surgery. Hence, in a preferred embodiment, the induced trauma is surgery. The use of the hemodynamic peptide for induced trauma, such as surgery, may be before, during and/or after surgery. It may be preferred that the use of the hemodynamic peptide, or an analog thereof, is during surgery. In particular, the surgery may more preferably require a cardiopulmonary bypass. Advantageously, the use of hemodynamic peptide as shown in the example section improved GFR in particular in patients having a long duration of cardiopulmonary bypass, and thus a long duration of infusion with hemodynamic peptide, i.e., longer than 2.5 hours. Hence, in a further embodiment, the use of the hemodynamic peptide, or an analog thereof, is during a cardiopulmonary bypass of longer than 2.5 hours and wherein the hemodynamic peptide or (functional) analog thereof is administered during the cardiopulmonary bypass. Preferably, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours or at least 2.5 hours, more preferably at least 3.5 hours, more preferably at least 4.5 hours. Typically the administration of any hemodynamic peptide (or combination thereof) according to this disclosure may last the whole of the intervention procedure and sometime thereafter. It is however possible to determine during the intervention whether modulating the fluid balance in the subject treated requires administration of a composition or formulation according to this disclosure and start the administration during the intervention. In another, or further, embodiment, the use of a hemodynamic peptide, or a functional analog thereof, for use in accordance with this disclosure is for use is in a human subject having heart failure. Preferably, in use in a human subject having heart failure, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours, more preferably at least 3.5 hours, more preferably at least 4.5 hours.

In another embodiment, the use of hemodynamic peptide, or a functional analog thereof, in accordance with this disclosure, is not restricted to patients having kidney injury and/or requiring hemodynamic therapy. This disclosure includes the use of a hemodynamic peptide, or a functional analog thereof, for use in the treatment of a human subject considered at risk or suffering from fluid overload, the use comprising modifying hemodynamics in the human subject. The use of hemodynamic peptide, or a functional analog thereof, in accordance with this disclosure, includes the treatment of human patients that are believed to be at risk of having fluid overload and/or anticipated to require hemodynamic therapy. Such human patients include patients that are to be admitted, or are expected to be admitted, into intensive care. Hence, the use of hemodynamic peptide, or a functional analog thereof, includes a use for prevention of induced fluid overload, such as with fluid therapy, as shown e.g., in the examples. Preferably, in use for prevention of induced fluid overload, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, or at least 2.5 hours, more preferably at least 3.5 hours, more preferably at least 4.5 hours.

In another embodiment, the use of hemodynamic peptide, or a functional analog thereof, in accordance with this disclosure, is not restricted to patients having kidney injury and/or requiring hemodynamic therapy. This disclosure includes the use of a hemodynamic peptide, or a functional analog thereof, for use in the treatment of a human subject considered to need vasopressor/inotropic treatment, the use comprising modifying hemodynamics in the human subject. The use of hemodynamic peptide, or a functional analog thereof, in accordance with this disclosure, includes the treatment of human patients that are believed to be at risk because of treatment with a vasopressor or an inotropic medication and/or anticipated to require hemodynamic therapy. Such human patients include patients that are to be admitted, or are expected to be admitted, into intensive care. Hence, the use of hemodynamic peptide, or a functional analog thereof, includes a use for the treatment of human patients that are believed to be at risk from treatment with vasopressor or inotropic use, such as treatment with medication selected from the group of dopamine, dobutamine, adrenaline, noradrenaline, phenylephrine, vasopressin and milrinone, as shown e.g., in the examples. Preferably, in use for the treatment of human patients that are believed to be at risk from treatment with vasopressor or inotropic use, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours, more preferably at least 3.5 hours, more preferably at least 4.5 hours.

In another embodiment, the use of hemodynamic peptide, or a functional analog thereof, in accordance with this disclosure, is not restricted to patients having kidney injury and/or requiring hemodynamic therapy. This disclosure includes the use of a hemodynamic peptide, or a functional analog thereof, for use in the treatment of a human subject to improve the subject's length of stay at the ICU, further to shorten the subject's length of stay at the ICU, the use comprising modifying hemodynamics in the human subject. The use of hemodynamic peptide, or a functional analog thereof, in accordance with this disclosure, includes the treatment of human patients that are believed to be at risk from treatment with a vasopressor or an inotropic medication and/or anticipated to require hemodynamic therapy with fluid therapy. Such human patients include patients that are or are to be admitted, or are expected to be admitted, into intensive care, and for which shortening length-of-stay at ICU is desired. Hence, the use of hemodynamic peptide, or a functional analog thereof, includes a use for the treatment of human patients that are believed to be at risk from treatment with vasopressor or inotropic medication and/or with fluid therapy, is provided as shown e.g., in the examples. Preferably, in use for shortening a subject's length of stay at the ICU, in human patients that are believed to be at risk, in particular upon entering at the ICU, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours, more preferably at least 3.5 hours, more preferably at least 4.5 hours.

In another embodiment, the use of hemodynamic peptide, or a functional analog thereof, in accordance with this disclosure, is not restricted to patients having kidney injury and/or requiring hemodynamic therapy. This disclosure includes the use of a hemodynamic peptide, or a functional analog thereof, for use in the treatment of a human subject to improve (i.e., reduce) the subject's length of stay at the hospital, the use comprising modifying hemodynamics in the human subject. The use of hemodynamic peptide, or a functional analog thereof, in accordance with this disclosure, includes the treatment of human patients that are believed to be at risk from treatment with and/or are expected to need a vasopressor or an inotropic medication and/or anticipated to require hemodynamic therapy with fluid therapy. Such human patients include patients that are or are to be admitted, or are expected to be admitted, into intensive care or hospital, and for which shortening length-of-stay at hospital is desired. Hence, the use of hemodynamic peptide, or a functional analog thereof, includes a use for the treatment of human patients that are believed to be at risk from treatment with vasopressor or inotropic medication and/or with fluid therapy, is provided as shown e.g., in the examples. Preferably, in use for shortening a subject's length of stay at the ICU, in human patients that are believed to be at risk, in particular upon entering at the ICU, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours, more preferably at least 3.5 hours, more preferably at least 4.5 hours.

This disclosure also provides a hemodynamic peptide for use in treatment of a human subject considered in need of maintaining or improving hemodynamic stability, the hemodynamic peptide comprising at least 50% amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, the hemodynamic peptide comprises at least 50%, more preferably at least 60%, most preferably at least 70% amino acids selected from the group of autophagy inhibiting amino acids alanine (A), glutamine (Q), and leucine (L), which were found to have best autophagy inhibiting characteristics. In a more preferred embodiment, the hemodynamic peptide comprises at most 30%, more preferably at most 20%, most preferably at most 10% amino acids selected from the group of autophagy inhibiting amino acids glycine (G), valine (V), isoleucine (I), proline (P) and arginine (R), inclusion of which is desirable to render proteolytic susceptibility characteristics to a hemodynamic peptide, if desired. In one embodiment, it is preferred that the hemodynamic peptide comprises amino acids selected from the group of autophagy inhibiting amino acids alanine (in one letter code: A), glutamine (Q), glycine (G) and valine (V). For improving solubility characteristics, it is preferred that the hemodynamic peptide is a salt selected from the group of hemodynamic peptide-acetate, more preferably hemodynamic peptide-tartrate, most preferably hemodynamic peptide-citrate. It is preferred that a hemodynamic peptide varies in length from 4-30 amino acids.

This disclosure also providers a hemodynamic peptide for use in treatment of a human subject considered in need of reducing adverse vascular permeability, the hemodynamic peptide comprising at least 50% amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, the hemodynamic peptide comprises at least 50%, more preferably at least 60%, most preferably at least 70% amino acids selected from the group of autophagy inhibiting amino acids alanine (A), glutamine (Q), and leucine (L), which were found to have best autophagy inhibiting characteristics. In a more preferred embodiment, the hemodynamic peptide comprises at most 30%, more preferably at most 20%, most preferably at most 10% amino acids selected from the group of autophagy inhibiting amino acids glycine (G), valine (V), isoleucine (I), proline (P) and arginine (R), inclusion of which is desirable to render proteolytic susceptibility characteristics to a hemodynamic peptide, if desired. In one embodiment, it is preferred that the hemodynamic peptide comprises amino acids selected from the group of autophagy inhibiting amino acids alanine (in one letter code: A), glutamine (Q), glycine (G) and valine (V). For improving solubility characteristics, it is preferred that the hemodynamic peptide is a salt selected from the group of hemodynamic peptide-acetate, more preferably hemodynamic peptide-tartrate, most preferably hemodynamic peptide-citrate. It is preferred that a hemodynamic peptide varies in length from 4-30 amino acids.

This disclosure also provides a hemodynamic peptide for use in treatment of a human subject considered in need of reducing adverse fluid, the hemodynamic peptide comprising at least 50% amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, the hemodynamic peptide comprises at least 50%, more preferably at least 60%, most preferably at least 70% amino acids selected from the group of autophagy inhibiting amino acids alanine (A), glutamine (Q), and leucine (L), which were found to have best autophagy inhibiting characteristics. In a more preferred embodiment, the hemodynamic peptide comprises at most 30%, more preferably at most 20%, most preferably at most 10% amino acids selected from the group of autophagy inhibiting amino acids glycine (G), valine (V), isoleucine (I), proline (P) and arginine (R), inclusion of which is desirable to render proteolytic susceptibility characteristics to a hemodynamic peptide, if desired. In one embodiment, it is preferred that the hemodynamic peptide comprises amino acids selected from the group of autophagy inhibiting amino acids alanine (in one letter code: A), glutamine (Q), glycine (G) and valine (V). For improving solubility characteristics, it is preferred that the hemodynamic peptide is a salt selected from the group of hemodynamic peptide-acetate, more preferably hemodynamic peptide-tartrate, most preferably hemodynamic peptide-citrate. It is preferred that a hemodynamic peptide varies in length from 4-30 amino acids.

This disclosure also provides a hemodynamic peptide for use in treatment of a human subject suffering or considered from Clarkson's disease (CLS), the hemodynamic peptide comprising at least 50% amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, the hemodynamic peptide comprises at least 50%, more preferably at least 60%, most preferably at least 70% amino acids selected from the group of autophagy inhibiting amino acids alanine (A), glutamine (Q), and leucine (L), which were found to have best autophagy inhibiting characteristics. In a more preferred embodiment, the hemodynamic peptide comprises at most 30%, more preferably at most 20%, most preferably at most 10% amino acids selected from the group of autophagy inhibiting amino acids glycine (G), valine (V), isoleucine (I), proline (P) and arginine (R), inclusion of which is desirable to render proteolytic susceptibility characteristics to a hemodynamic peptide, if desired. In one embodiment, it is preferred that the hemodynamic peptide comprises amino acids selected from the group of autophagy inhibiting amino acids alanine (in one letter code: A), glutamine (Q), glycine (G) and valine (V). For improving solubility characteristics, it is preferred that the hemodynamic peptide is a salt selected from the group of hemodynamic peptide-acetate, more preferably hemodynamic peptide-tartrate, most preferably hemodynamic peptide-citrate. It is preferred that a hemodynamic peptide varies in length from 4-30 amino acids.

This disclosure also provides a pharmaceutical formulation according to this disclosure comprising at least two different hemodynamic peptides each comprising at least 50% amino acids selected from the group of autophagy inhibiting amino acids alanine (in one letter code: A), glutamine (Q), glycine (G), valine (V), leucine (L), isoleucine (I), proline (P) and arginine (R). In a preferred embodiment, the hemodynamic peptide comprises at least 50%, more preferably at least 60%, most preferably at least 70% amino acids selected from the group of autophagy inhibiting amino acids alanine (A), glutamine (Q), and leucine (L), which were found to have best autophagy inhibiting characteristics. In a more preferred embodiment, the hemodynamic peptide comprises at most 30%, more preferably at most 20%, most preferably at most 10% amino acids selected from the group of autophagy inhibiting amino acids glycine (G), valine (V), isoleucine (I), proline (P) and arginine (R), inclusion of which is desirable to render proteolytic susceptibility characteristics to a hemodynamic peptide, if desired. In one embodiment, it is preferred that the hemodynamic peptide comprises amino acids selected from the group of autophagy inhibiting amino acids alanine (in one letter code: A), glutamine (Q), glycine (G) and valine (V). For improving solubility characteristics, it is preferred that the hemodynamic peptide is a salt selected from the group of hemodynamic peptide-acetate, more preferably hemodynamic peptide-tartrate, most preferably hemodynamic peptide-citrate. It is preferred that a hemodynamic peptide varies in length from 4-30 amino acids. Furthermore, it is preferred that the pharmaceutical formulation comprises at least 0.85 mol/L of the hemodynamic peptide or hemodynamic peptides. In another embodiment, the pharmaceutical formulation comprises at least one pharmaceutically acceptable excipient. Examples of such formulations are stock solutions of a hemodynamic peptide as provided herein.

This disclosure also provides use of a formulation or solution according to this disclosure for use in a method of treatment of a human subject suffering or considered suffering from Clarkson's disease (CLS), in a method of treatment of a human subject considered in need of maintaining or improving hemodynamic stability, a method of treatment of a human subject considered in need of reducing adverse vascular permeability, in a method of treatment of a human subject considered in need of reducing adverse fluid retention.

Preferably, the use of the hemodynamic peptide, or a functional analog thereof, in accordance with this disclosure and as described above, involves the administration of the peptide into the bloodstream. It is understood that administration into the bloodstream comprises e.g., intravenous administration or intra-arterial administration. A constant supply of hemodynamic peptide, or an analog thereof, is preferred, e.g., via an infusion wherein the hemodynamic peptide, or analog thereof, is comprised in a physiological acceptable solution. Suitable physiological acceptable solutions may comprise physiological salt solutions (e.g., 0.9% NaCl) or any other suitable solution for injection and/or infusion. Such physiological solutions may comprise further compounds (e.g., glucose etc.) that may further benefit the human subject, and may also include other pharmaceutical compounds (e.g., vasopressors, typically at a reduced rate).

Preferably, the hemodynamic peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 70 mg/kg/hr and administered for at least 1 hour, more preferably at least 1.5 hours, most preferably at least 2 hours, such as at least 2.5 hours, more preferably at least 3.5 hours, more preferably at least 4.5 hours. Preferably, the administration is during surgery. More preferably, the administration is during the entire duration of surgery.

As shown in the example section, the mean arterial maximum concentrations (mean Cmax) as determined in vivo in humans for EA-230 in the Phase II clinical trial was 30500 ng/ml, in the range of 12500 to 57500 ng/ml. The mean venous Cmax found was 68400 ng/ml, in the range of 19600 to 113000 ng/ml. Hence, whichever means and methods are used for administration of EA-230 (or AQGV (SEQ ID NO:1)), preferably, means and methods that allow to obtain an arterial Cmax in the range of 10,000 to 60,000 ng/ml and/or a venous Cmax in the range of 15000 to 120000 ng/ml can be contemplated. Thus, the route of administration may not be necessarily be restricted to intravenous administration, but may include other routes of administration resulting in similar venous and/or arterial Cmax concentrations.

In another embodiment, a hemodynamic peptide, or a functional analog thereof, is provided for any use in accordance with this disclosure as described above, wherein the human subject is admitted to intensive care, and wherein the use improves parameters measured of the human subject, the parameters of the human subject determined to assess to remain in intensive care or not. As shown above, parameters that are assessed when a human patient is in intensive care include parameters related to kidney function and hemodynamics. In any case, the use of the hemodynamic peptide, or analog thereof, is to improve such parameters to thereby reduce the length of stay in the intensive care unit. Not only does the use of the hemodynamic peptide, or analog thereof reduce the length of stay in the intensive care, the effect of the use of the hemodynamic peptide, or analog thereof, also reduces the length of stay in the hospital and reduces readmittance into the hospital.

In any case, the use of the hemodynamic peptide, or a functional analog thereof has a profound effect on kidney function and/or hemodynamics in human subjects thereby advantageously benefiting human subjects when e.g., suffering from induced trauma, e.g., when undergoing cardiac surgery and being on a cardiopulmonary bypass pump. Hence, in one embodiment, the use of the hemodynamic peptide, or a functional analog thereof, is for use in cardiac surgery. In another embodiment, the use of the hemodynamic peptide, or a functional analog thereof, is for use in human patients being on a cardiopulmonary bypass pump.

Without being bound by theory, the effect of the hemodynamic peptide, or a functional analog thereof, may have an effect on vasoconstriction. Vasoconstriction involves the narrowing of the blood vessels resulting from contraction of the muscular wall of the vessel. Hence, in one embodiment, the use of a hemodynamic peptide, or a functional analog thereof, in accordance with this disclosure, involves inducing vasoconstriction. In particular, the use of AQGV (SEQ ID NO:1), or an analog thereof, may induce peripheral vasoconstriction and/or vasoconstriction in efferent arterioles of the kidney. Peripheral vasoconstriction may improve hemodynamics, whereas vasoconstriction in efferent arterioles may improve kidney function.

### BRIEF DESCRIPTION OF THE DRAWINGS

FIG. 1. An overview of the timeline with procedures of the EASI-study (JMIR Res. Protol. 2019 Feb. 6; 8(2):e11441. doi: 10.2196/11441) from inclusion until end of follow-up. The EASI-study is a prospective, randomized, double-blind, placebo-controlled study in which 180 elective patients undergoing on-pump coronary artery bypass grafting (CABG) with or without concomitant valve surgery were enrolled. Patients were randomized in a 1:1 ratio to receive either EA-230 (SEQ ID NO:1), 90 mg/kg/hour, or placebo, infused from the start of the surgical procedure until the end of the use of the cardiopulmonary bypass (CBP). 89 patients received placebo, 91 patients received EA 230, administered i.v. via 2-4 hour continuous i.v. infusion.

FIG. 2. Depicted is the need for vasopressors in the first 24 hours of intensive care unit (ICU) after the surgery. In the total group (FIG. on top) and related to treatment duration. As study drug infusion was continued as long as the patient was on cardiopulmonary bypass, treatment duration was variable (figures below). Modulation in vasopressor use during the first 24 hours of ICU admission were determined and expressed as the composite inotropic score: (dopamine dose×1 μg/kg/min)+(dobutamine dose×1 μg/kg/min)+(adrenaline dose×100 μg/kg/min)+(noradrenaline dose×100 μg/kg/min)+(phenylephrine dose×100 μg/kg/min)+(vasopressin (mUnits/kg/min)*10000)+(milrinone×10 mcg/kg/min) (Pediatric critical care medicine: a journal of the Society of Critical Care Medicine and the World Federation of Pediatric Intensive and Critical Care Societies. 2010 March; 11(2):234-8. PMID: 19794327. doi: 10.1097/PCC.0b013e3181b806fc.) Data was log-transformed for repeated measures two-way ANOVA analysis.

FIGS. 3A and 3B. Effects of EA-230 (SEQ ID NO:1) on the incidence of different stages of acute kidney injury (AKI) were determined according to the RIFLE criteria (RIFLE: risk, injury, failure, loss of kidney function, and end-stage kidney disease classification, Clin. Kidney J. 2013 February; 6(1): 8-14). In the EA-230 group, the percentage of patients with no AKI increased, whereas the percentage of patients in the Injury category of the RIFLE criteria decreased. In FIG. 3A results are given on full RIFLE score, in FIG. 3B, results are given grouped on creatinine- and GFR-data only.

FIG. 4. Effects of EA-230 (SEQ ID NO:1) on glomerular filtration rate (GFR) were determined by MDRD (Am. J. Kidney Dis. 2002 February; 39 (2 Suppl 1):S1-266). Treatment with EA-230 significantly improved GFR after surgery (day+1) when compared with GFR before surgery (day −1), where treatment with placebo did not, (left). At right, it is shown that MDRD effects converge after day 1, when treatment with EA-230 had stopped.

FIG. 5. Effects of EA-230 (SEQ ID NO:1) on plasma creatinine concentrations as a biomarker of kidney function. Treatment with EA-230 significantly improved creatinine levels after surgery (day+1) when compared with day −1, (before surgery), where treatment with placebo does not.

FIG. 6. EA-230 effects on plasma creatinine related to pre-surgery kidney function. When baseline kidney function was below 60 ml/min/1.73 m2, EA-230 (SEQ ID NO:1) significantly (p=0.012) improved creatinine levels, when kidney function was above 60 ml/min/1.73 m2, no statistically significant differences were found between groups. RM 2 way ANOVA.

FIG. 7. Effects of EA-230 (SEQ ID NO:1) on GFR (MDRD) related to pre-surgery kidney function. When baseline kidney function was below 60 ml/min/1.73 m2, treatment with EA-230 significantly (p=0.021) improved estimated GFR after surgery (day+1), when compared with estimated GFR before surgery (day −1), where treatment with placebo did not. When kidney function was above 60 ml/min/1.73 m2, no statistically significant differences were found between groups. RM 2 way ANOVA.

FIG. 8. Effects of EA-230 (SEQ ID NO:1) on MDRD related to duration of cardiac-pulmonary bypass (CPB) and thus duration of study drug infusion. Treatment with EA-230 significantly improved GFR after surgery (day+1) in the group of patients having long duration of cardio-pulmonary bypass (and thus longer study drug infusion) (>median length), when compared with GFR before surgery (day −1), where treatment with placebo did not. When CPB duration (and thus study drug infusion duration) was short (<median length), no differences were found between groups. RM 2 way ANOVA.

FIG. 9. No immunomodulatory effects of EA-230 (SEQ ID NO:1). EA-230 was well tolerated and showed an excellent safety profile. However, treatment with EA-230 did not result in a significant change of the primary endpoint plasma IL-6. Here results of IL-6 testing are shown in the full set of patients having short or long duration of cardio-pulmonary bypass.

FIG. 10. Effects of EA-230 (SEQ ID NO:1) on length of stay. In the EASI-study, effects on length of stay in the ICU of patients and length of stay in the hospital (inpatient care) were investigated. Treatment with EA-230 resulted in a significant reduction of the length of stay (LOS) in the ICU, as well as in the hospital. LOS in the ICU and the hospital was reduced in the EA-230 group: 24 hours after ICU admission, 12% of patients of the EA-230 group were in ICU versus 22% in the control group (p=0.02) and in-hospital stay was 195 [171-265] and 234 [192-295] hours in the EA-230 and placebo groups, respectively (p=0.002). The patients treated with EA-230 also showed a considerable (p=0.09) reduction of the number of re-admissions to the hospital up to 90 days after surgery.

FIG. 11. Correlation between length of stay in hospital and kidney injury stage as assessed with the RIFLE score per treatment group (placebo or AQGV (SEQ ID NO:1)). In general, and accordance with literature, more severe kidney injury is associated with a prolonged length of stay, as observed here in the placebo group. While for the placebo group, this association was observed, in the AQGV group no such association was present, suggesting that AKI is resolving more quickly. In conclusion, in the patient group treated with AQGV, the number of patients suffering from AKI Injury was reduced, and when patients did develop AKI, these patients did not have a prolonged hospital length-of-stay, as observed in the placebo group and length of stay was similar to patients having no AKI or patients being at risk of AKI.

FIG. 12. Treatment with EA-230 (SEQ ID NO:1) overall did not result in a significant change of the primary endpoint plasma IL-6. Here results of IL-6 testing are shown in the full set of patients having short or long duration of cardio-pulmonary bypass.

FIG. 13. Treatment with EA-230 (SEQ ID NO:1) overall did not result in a significant change of plasma IL-8, IL-10, IL-1RA, IL-17, MIP-1a, MIP-1b, MCP-1 ICAM, VCAM, and other cytokines tested, no immunomodulatory effects were observed. Treatment with EA-230 did not show effects on clearance of iohexol.

FIG. 14. The need for treatment of hemodynamic instability by use of vasopressors after the surgery (left) and by use of fluid therapy to adjust net fluid balance were considerably improved in the first 24 hours of intensive care unit (ICU) in those patients given EA-230 peptide. Therewith, EA-230 (SEQ ID NO:1) given during surgery significantly improves hemodynamic recovery after surgery, providing a significant improvement of hemodynamic stability (reducing a composite measure of required fluid therapy and blood pressure medication; 2-way ANOVA; p=0.006).

FIG. 15. Treatment of CABG patients with EA-230 (SEQ ID NO:1) during surgery resulted in a highly significant and nearly 40% shorter length-of-stay in the Intensive Care Unit (ICU). Where, on average, placebo-treated CABG patients required 40 hours at ICU, those treated with EA-230 were already cleared to go after 25 hours; freeing valuable ICU space for others.

FIG. 16. Patients treated with EA-230 (SEQ ID NO:1) show a statistical highly significant reduction of over 20% of length-of-stay in the hospital. Where, on average, placebo-treated patients required nearly 12 hospital days of continued care to recover from CABG-surgery, those treated with EA-230 recovered and left the hospital 2.5 days faster. In conclusion, treatment with EA-230 resulted in a highly significant reduction in post-operative length-of-stay in patients undergoing elective CABG surgery. Moreover, this beneficial reduction of length-of-stay did not increase re-admission risk; EA 230 resulted in considerably lower risks for 90-day re-admission (4 for EA-230, 10 for placebo; p=0.09) instead.

FIG. 17. Beneficial effects of prolonged treatment (long=longer than median) with EA-230 (SEQ ID NO:1) on kidney function, measured as glomerular filtration rate (GFR), as compared with patients that were treated shorter than median treatment. Findings indicate highly significant statistical improvements of kidney function after surgery due to prolonged treatment of patients with EA-230 during elective surgery.

FIG. 18. Beneficial effects of prolonged treatment (long=longer than median) with EA-230 (SEQ ID NO:1) on post-operative hemodynamic stability (measured as need for vasopressor/inotropic or fluid therapy) after surgery, as compared with patients that were treated shorter than median treatment. Hemodynamic stability benefits significantly from prolonged use of EA-230 during surgery (POD=post-operative day).

FIG. 19. Overview of efficacy endpoints results in inflammation, renal, cardiovascular and general. (a) Inflammatory. Left panel: Plasma concentrations of interleukin (IL)-6 over time from pre-operative time point (baseline) until the next postoperative morning (POM) (p=0.99). Blue box indicates the period in which study drug was administered. Right panel: Area under the plasma concentration-time effect curve (AUEC) of IL-6. Data presented as median and interquartile range. P-values calculated using repeated measures two-way analysis of variance (ANOVA, interaction term, left panel) or Mann-Whitney U test (right panel). (b) Renal. Left panel: Renal function expressed as GFRiohexol and eGFRMDRD from the day before surgery (baseline) until the next POM. Data presented as mean and standard error of the mean. P-values calculated using repeated measures two-way analysis of variance (ANOVA, interaction term). Right panel: Classification of acute kidney injury (AKI) according to the RIFLE criteria; patients were classified as “no AKI” (n=50 in the EA-230 group, n=42 in the placebo group), “Risk” (n=34 in the EA-230 group, n=31 in the placebo group) or “Injury” (n=6 in the EA-230 group, n=16 in the placebo group), no patients were classified as stage “Failure,” “Loss of function” or “End stage of renal disease.” Data presented as percentages of patients. P-value calculated using Pearson's chi-square test. (c) Cardiovascular. Left panel: Net fluid balance during the first 24 hours after Intensive Care Unit (ICU) admission (p=0.97). Right panel: Cumulative postoperative net fluid balance on postoperative day (POD) 1 (n=90 in the EA-230 group and n=89 in the placebo group), on POD 2 (n=90 in the EA-230 group and n=89 in the placebo group) and on POD 3 (n=86 in the EA-230 group and n=85 in the placebo). POD 4-7 not depicted due to few available data. Data presented as mean and standard error of the mean. P-values calculated using repeated measures two-way analysis of variance (ANOVA, interaction term, left panel) or Student's t-tests (right panel). (d) General. Left panel: Length of Stay in the ICU (p=0.02). Right panel Length of stay in the hospital (p=0.001). P-values calculated using log-rank test. CPB: cardiopulmonary bypass; (e) GFR: (estimated) glomerular filtration rate; MDRD: modification of diet in renal disease; pg: picograms; ml: milliliters; h: hour; min: minute; m: meter.

FIG. 20. Post-hoc analyses using the subgroups short (n=90) and long (n=89) surgery duration (divided using median). (a) Area under the plasma concentration-time effect curve (AUEC) of interleukin (IL)-6 plasma concentrations tested between treatment groups (short: EA—230 vs Placebo: p=0.88 and long: EA—230 vs Placebo: p=0.41). Data presented as median and interquartile range (IQR). (b) Net fluid balance per day (short: p=0.54, p=0.33, p=0.75 and p=0.84 for first Intensive Care Unit (ICU) day, postoperative day (POD) 1, POD 2 and POD 3, respectively. Long: p=0.09, p=0.008, p=0.09 and p=0.89 for first ICU day, POD 1, POD 2 and POD 3, respectively). Data presented as mean and standard error of the mean (SEM). (c): Vasoactive and inotropic agents administered during the first 24 hours of the ICU admission depicted as the Inotropic Score (short: p=0.28, long: p=0.048). Data presented as median and IQR. (d and e): Renal function depicted as GFRiohexol (d) and eGFRmdrd (e) (short: GFRiohexol: p=0.47 and eGFRmdrd: p=0.27, long: GFRiohexol: p=0.02 and eGFRmdrd: p<0.0001). Data is presented as mean and SEM. P-values calculated using Mann-Whitney U or Student's t-tests. *: p<0.05; #: p<0.1; h: hour.

FIG. 21. Renal function parameters. The blue area depicts the period of study drug administration. (a) Plasma concentrations of Creatinine (p=0.022) and corresponding eGFRMDRD (p=0.663) (b) from baseline (day before surgery) until the seventh postoperative day (POD). Baseline and POD 1 are samples collected for this study, the measurements of POD 2-7 are additional samples extracted from the Electronic Patient Records and were not available daily in all patients: n=60, 48, 68, 21, 21, 17 for POD 2, 3, 4, 5, 6, and 7, respectively. (c) Renal function depicted as Endogenous Creatinine Clearance (GFRECC) (p=0.74). ¬Sample collection for this parameter was performed from start of surgery until the first postoperative day in the morning. (d) Urinary creatinine (p=0.029) and (e) urea (p=0.004) concentrations. (f) Proenkephalin plasma concentrations (p=0.53). Data of (a), (b) and (c) presented as mean±standard error of the mean. Data of (d), (e) and (f) are presented as median with interquartile range. P-values of (a), (b), (d), (e) and (f) calculated using repeated measures two-way analysis of variance (ANOVA, interaction term). P-value of (c) calculated using a Student's t-test. CPB: cardiopulmonary bypass; POD: postoperative day; h: hours; (e) GFR: (estimated) glomerular filtration rate derived with the modification of diet in renal disease formula.

FIG. 22. Urinary renal injury marker per millimole (mmol) creatinine (Cr) over time from pre-operative time point (baseline) until the next post-operative morning (POM) of (a) interleukin (IL)-18 (p=0.78), (b) kidney injury molecule (KIM)1 (p=0.21), (c) neutrophil gelatinase-associated lipocalin (NGAL) (p=0.92), (d) liver-type fatty acid-binding protein (L-FABP) (p=0.23), (e) n-acetyl-beta-d-glucosaminidase (NAG) (p=0.14). The blue box indicates the period in which study drug was administered. Data presented as median with interquartile range. P-values calculated using repeated measures two-way analysis of variance (ANOVA, interaction term). ng: nanograms; CPB: cardiopulmonary bypass; μg: micrograms; h: hour

FIG. 23. Overview of solubility experiments with results.

FIG. 24. Based on the results depicted in FIG. 22, the concentration below which an aggregated peptide-salt tends to resolve of the neutral-peptides salts screened were determined (aggregation points). It can be concluded that changing the anion significantly influences the solubility characteristics of AQGV (SEQ ID NO:1). Higher solubility (solubility in 0.9% NaCl) and therewith higher aggregation points were observed for the AQGV-citric acid (AQGV (SEQ ID NO:1)-citrate) and -tartaric acid (AQGV (SEQ ID NO:1)-tartrate) salt, whereas maleic acid and KHSO4 salts showed lower solubility, compared to AQGV-Ac. Using adenosine-monophosphate or adenosine did not provide solubility. Citric acid seems to be a special case. Highly concentrated solution does not crystallize or aggregate but tend to form a highly viscous solution.

### DETAILED DESCRIPTION

Peptide Synthesis

Hemodynamic peptides are for example synthesized using classical solid phase synthesis, or other methods known in the art. Purity of the peptides is confirmed by high performance liquid chromatography and/or by fast atom bombardment mass spectrometry. Traditionally, peptides are defined as molecules that consist of between 2 and 50 amino acids, whereas proteins are made up of 50 or more amino acids. In addition, peptides tend to be less well defined in structure than proteins, which can adopt complex conformations known as secondary, tertiary, and quaternary structures. Functional distinctions may also be made between peptides and proteins. In fact, most researchers, as well as this disclosure, use the term peptide to refer specifically to peptides, or otherwise relatively short amino acid chains of up to 50 amino acids, with the term polypeptide being used to describe proteins, or chains of >50 or many more amino acids.

Peptide Administration

As shown in clinical trial protocol (Groenendael et al., JMIR Res Protoc 2019 February; 8(2): e11441), study medication EA-230 formulation is packed and provided in sterile 5-mL glass vials, containing 1500 mg/vial, dissolved in water for injection at a final concentration of 300 mg/mL with an osmolality of 800 to 1000 mOsm/kg. The placebo formulation comprises sodium chloride diluted in water for injection in identical sterile 5-mL glass vials containing 29 mg/mL to reach a solution with an identical osmolality. EA-230 and placebo are prepared for continuous intravenous infusion with an osmolality of <400 mOsm/kg by adding the appropriate amount of EA-230 or placebo to 1000 mL normal saline under aseptic conditions.

Need for Stock-Solution with Higher Concentration Active Substance.

A vial with EA-230 formulation (stock-solution) used in herein referenced clinical trial contained 1.5 gram EA-230, each vial containing 5 ml a 300 mg/ml [(300 g/L=0.8 mol/L) AQGV (SEQ ID NO:1) having a molecular weight of 373 g/mol]. In the trial, a best-treatment practice was established when infusion with active substance lasted at least 1.5 hours, preferably at least 2.5 hours, preferably at least, 3.5 hours, more preferably at least 4.5 hours, at 90 mg/kg per hour. As a consequence, and also depending on bodyweight, often more than 12-17 vials were needed for continuing effective treatment, an administration requirement that takes (too) much labor in the operating room or ICU for the required care. This disadvantage of treatment with too weak amounts of stock of EA-230 formulation brings forward a need to provide more and better concentrated stock-solutions than available.

Determination of Aggregation Points

It is recognized herein that many drug-like molecules can self-aggregate in aqueous media and aggregates may have physicochemical properties that skew experimental results and clinical decisions. The aggregation of peptide drugs is one of the most common and troubling processes encountered in almost all phases of biological drug development. Aggregation can take several different forms and the term is used to describe a number of different processes during which peptide molecules associate into larger species comprising multiple polypeptide chains. Aggregates can be amorphous or highly structured, e.g., amyloid fibrils, and can form in solution or on surfaces due to adsorption. They can arise as a result of the non-covalent association of polypeptide chains, or from covalent linkage of chains. In some cases, aggregation is reversible while in others it is effectively irreversible. In either case, it reduces the physical stability of the peptide in question, not only leading to a loss in activity but also other critical problems such as toxicity and immunogenicity.

Salts have complex effects on the physical stability of biomolecules affecting both conformational and colloidal stability. Their effects frequently vary according to the surface charge on the peptide and the overall effect of a salt on physical stability is a balance of different and multiple mechanisms by which salt interacts with water and biomolecules. Various salts can influence physical stability by altering the properties of the peptide-solvent system (Hofmeister effects) and by altering electrostatic interactions (Debye-Hückel effects).

One aim was to investigate the solubility of seven different salts on prototype autophagy inhibiting peptide AQGV (SEQ ID NO:1), using the modified shake flask method. At first, the AQGV-Ac salt will be converted to the free base, extracted with an organic solvent and concentrated in vacuo. Subsequently the citrate, maleate, sulfate (KHSO4), adenosine mono-phosphate, adenosine, acetate and tartaric acid salts will be prepared and screened for their solubility thereafter.

Results

Conversion to the Free Base.

Extraction of AQGV-Ac (SEQ ID NO:1-Ac) with organized solvents from neutralized solution (pH=6-7) turned out not to be possible. Therefore a solution of AQGV-Ac in water was transferred to an ion-exchange column (Amberlite, approximately 100 mL; IR120, H resin) The column was flushed using demi-water followed by 1N ammonia-solution. The first 3 basic fractions were concentrated to afford 4.7 g of the free base AQGV (SEQ ID NO:1; 1H-NMR).

Solubility Measurements.

At first attempts were made to mix a solution of the free base and an acid in order to achieve a concentrated DMSO solution of the salt and subsequently dilute this in water in order to determine solubility. However the salts attempted (adenosine and citric acid) did not dissolve in DMSO at all. In fact, the mixture became clear after the addition of a little water. Therefore the solubility determinations could not be conducted as planned originally. It was decided to determine the solubility of the salts required by dilution of known amount of salts (not soluble) till a clear solution is obtained.

For citric acid, 1 mmol AQGV (SEQ ID NO:1) and 1 mmol citric acid were mixed in 0.5 mL 0.9% NaCl. This afforded a clear solution. More material of both AQGV (SEQ ID NO:1) and citric acid were added (amounts of 0.5 and 0.25 mmol) until a total of 2.75 mmol was dissolved in the 0.5 mL 0.9% NaCl. The mixture remains clear but got very thick/viscous. The remaining experiments have been conducted differently: 1 or 0.5 mmol salt was weighed in a 4 ml vial and small amounts of 0.9% NaCl were added until a clear solution was obtained, which remained clear for more than a week. In case of adenosine and adenosine-monophosphate no clear solution could be obtained.

Based on the results depicted in Table 1 the concentration below which an aggregated peptide-salt tends to resolve of neutral and autophagy inhibiting peptide-salts screened were determined (aggregation points, see Table 2). It can be concluded that changing the anion significantly influences the solubility characteristics of AQGV (SEQ ID NO:1). Higher solubility (solubility in 0.9% NaCl) and therewith higher aggregation points were observed for the AQGV-citric acid (AQGV-citrate, >5.5 mol/L) and -tartaric acid (AQGV-tartrate) salt, whereas maleic acid and KHSO4 salts showed lower solubility, compared to AQGV-Ac (2 mol/L). Using adenosine-monophosphate or adenosine did not provide solubility. Citric acid seems to be a special case. Highly concentrated solution does not crystallize or aggregate but tend to form a highly viscous solution.

Heeding aggregation risk, a vial with a stock solution of hemodynamic peptide for use in a clinical trial hitherto contained no more than (0.8 mol/L) active substrate in solution. Based on this disclosure, such a stock solution of an AQGV (SEQ ID NO:1)-salt of an organic acid, in particular of AQGV peptide-maleate, AQGV peptide-acetate AQGV peptide-tartrate or AQGV peptide-citrate (but neither adenosine nor adenosine monophosphate) now is provided with or is prepared to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of the hemodynamic peptide-acetate, hemodynamic peptide tartrate or hemodynamic peptide-citrate. In a more preferred embodiment, this disclosure provides a stock-solution of the hemodynamic peptide-tartrate or the hemodynamic peptide-citrate wherein the concentration of the hemodynamic peptide is in the range of 2 mol/L to 2.5 mol/L. In a more preferred embodiment, this disclosure provides a stock-solution of the hemodynamic-peptide-citrate wherein the concentration of the peptide-citrate is in the range of 2.5 mol/L to 3 mol/L. In a more preferred embodiment, this disclosure provides a stock-solution of the peptide-citrate wherein the concentration of the peptide-citrate is in the range of 3 mol/L to 3.5 mol/L. In a more preferred embodiment, this disclosure provides a stock-solution of the peptide-citrate wherein the concentration of the peptide-citrate is in the range of 3.5 mol/L to 4.5 mol/L. In a more preferred embodiment, this disclosure provides a stock-solution of the peptide-citrate wherein the concentration of the peptide-citrate is in the range of 4.5 mol/L to 5.5 mol/L. In a more preferred embodiment, this disclosure provides a stock-solution of the peptide-citrate wherein the concentration of the peptide-citrate is equal to or larger than 5.5 mol/L. It is preferred that the stock solution is an aqueous solution.

In describing protein or peptide composition, structure and function herein, reference is made to amino acids. In the present specification, amino acid residues are referred to using the following abbreviations. Also, unless explicitly otherwise indicated, the amino acid sequences of peptides and proteins are identified from N-terminal to C-terminal, left terminal to right terminal, the N-terminal being identified as a first residue. Ala: alanine residue; Asp: aspartate residue; Glu: glutamate residue; Phe: phenylalanine residue; Gly: glycine residue; His: histidine residue; Ile: isoleucine residue; Lys: lysine residue; Leu: leucine residue; Met: methionine residue; Asn: asparagine residue; Pro: proline residue; Gln: glutamine residue; Arg: arginine residue; Ser: serine residue; Thr: threonine residue; Val: valine residue; Trp: tryptophane residue; Tyr: tyrosine residue; Cys: cysteine residue. The amino acids may also be referred to by their conventional one-letter code abbreviations; A=Ala; T=Thr; V=Val; C=Cys; L=Leu; Y=Tyr; I=Ile; N=Asn; P=Pro; Q=Gln; F=Phe; D=Asp; W=Trp; E=Glu; M=Met; K=Lys; G=Gly; R=Arg; S=Ser; and H=His.

Inhibition of Autophagy by Selected Amino Acids.

Autophagy is a degradation pathway that delivers extra cellular and cytoplasmic materials to lysosomes via double-membraned vesicles designated autophagosomes. Cytoplasmic constituents are sequestered into autophagosomes, which subsequently fuse with lysosomes, where the cargo is degraded. Extracellular materials are taken up by endocytosis or phagocytosis, which subsequently fuse with lysosomes, again where the cargo is degraded. Autophagy is a crucial mechanism involved in many aspects of cell function, including cellular metabolism and energy balance; and alterations in autophagy have been linked to various human pathological processes. Autophagy is a natural mechanism in which the cell removes and degrades cellular components with autolysosomes.

As recently reviewed (Cell. 2019 July; 8(7)), where the role of autophagy in the maintenance of tissue homeostasis is relatively well documented, its role during tissue repair and regeneration has only recently been appreciated. This disclosure provides that hemodynamic peptides, i.e., peptides enhanced with distinct amino acids or combinations thereof control the balance between on the one hand proteogenesis (mTOR kinase activities) and on the other hand proteolysis (autophagy) more than others, therewith identifying peptides enriched in autophagy inhibiting amino acids as better enhancers of proteogenesis underlying tissue repair than other peptides not being enriched in the amino acids. The mechanistic target of rapamycin complex I (mTORC1) is a central regulator of cellular and organismal growth and this pathway is implicated in the pathogenesis of many human diseases. mTORC1 promotes cell and tissue growth in response to the availability of nutrients, such as amino acids, which drive mTORC1 to the lysosomal surface, its site of activation. Recent and older data identify leucine (L), valine (V), isoleucine (I), alanine (A), glutamine (Q), arginine (R), glycine (G), proline (P), either alone or in combination, as more potent activators of mTOR or inhibitors of autophagy than other amino acids, such as glutamate (E), threonine (T), serine (S), lysine (K), threonine (T), phenylalanine (F), tyrosine (Y), and methionine (M) that have been reported to have no or opposite effects. Hence, as herein provided for inclusion in a hemodynamic peptide according to this disclosure, peptides enriched with leucine (L), valine (V), isoleucine (I), alanine (A), glutamine (Q), arginine (R), glycine (G), proline (P), either alone or (preferably) in combination, are most preferred activators of mTOR or inhibitors of autophagy for use in human cells, for packaging and targeting to cells. It is preferred that a hemodynamic peptide comprises at least 50%, more preferably at least 75% and most preferably 100% amino acids selected from the group A, Q, G, V, L, P, I and R. Preferably, a hemodynamic peptide as provided herein has a length in the range of 4-12 amino acids, more preferably 4-8 amino acids. Preferably, such a hemodynamic peptide is a linear peptide. A functional hemodynamic peptide analog according to this disclosure may be more preferably selected from the group of peptides comprising a dipeptide sequence selected from the group of AQ, LQ, PQ, VQ, GQ. A functional hemodynamic peptide according to this disclosure may be more preferably selected from the group of peptides comprising a tripeptide sequence selected from the group of AQL, LQL, PQL, VQL, GQL, PLQ, LQG, PQV, VGQ, LQP, LQV, AQG, QPL, PQV, VGQ, GQG. Amino acids leucine (L), alanine (A), glutamine (Q), and proline (P) are reported to have most prominent mTOR associated autophagic inhibitory effects on human cells (A J Meijer et al. Amino Acids 2015, 47, 2037-2063). Glycine (G; Zhong Z, Wheeler M D, Li X, Froh M, Schemmer P, Yin M, Bunzendaul H, Bradford B, Lemasters J J. 1-Glycine: a novel anti-inflammatory, immunomodulatory, and cytoprotective agent. Curr Opin Clin Nutr Metab Care 6: 229-240, 2003) improves amino-acid-stimulated mammalian target of rapamycin (mTOR) complex 1 activation. Hence, as herein provided for inclusion in a hemodynamic peptide according to this disclosure, leucine (L), alanine (A), glutamine (Q), glycine (G) and proline (P), either alone or (preferably) in combination, are preferred activators of mTOR or inhibitors of autophagy for use in human cells. It is more preferred that a hemodynamic peptide comprises at least 50%, more preferably at least 75% and most preferably 100% amino acids selected from the group A, Q, G, V, L, and P. In a more preferred embodiment, a hemodynamic peptide comprises at least 75% and most preferably 100% amino acids selected from the group A, Q, G, and V.

In a most preferred embodiment, a hemodynamic peptide according to this disclosure is a tetrapeptide that comprises 100% amino acids selected from the group A, Q, G, and V. Typical preferred examples of such a preferred tetrapeptide are AQGV (SEQ ID NO:1), LQGV (SEQ ID NO:4), VGQA (SEQ ID NO:3), VGQL (SEQ ID NO:6), AQVG (SEQ ID NO:2), and LQVG (SEQ ID NO:5). Most typically preferred is AQGV (SEQ ID NO:1) having been subject of a human clinical trial as provided below.

This disclosure includes the use of a hemodynamic peptide, or a functional analog thereof, for use in the treatment of a human subject to improve the subject's length of stay at the ICU, further to shorten the subject's length of stay at the ICU. One way in which this may be attained is by modifying fluid retention in the human subject. The use of hemodynamic peptide, or a functional analog thereof, in accordance with this disclosure, includes the treatment of human patients that are believed to be at risk from treatment with a vasopressor or an inotropic medication and/or anticipated to require hemodynamic therapy with fluid therapy. Such human patients include patients that are or are to be admitted, or are expected to be admitted, into intensive care, and for which shortening length-of-stay at ICU is desired. Hence, the use of hemodynamic peptide, or a functional analog thereof, includes a use for the treatment of human patients that are believed to be at risk from treatment or expected to need treatment with vasopressor or inotropic medication and/or with fluid therapy, is provided as shown e.g., in the examples. Preferably, in use for shortening a subject's length of stay at the ICU, in human patients that are believed to be at risk, the hemodynamic peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the hemodynamic peptide is administered for at least 1 hour, more preferably at least 1.5 hours, most preferably at least 2 hours. Preferably, the administration of the hemodynamic peptide is at a rate of at least 20 mg/kg/hr and administered for at least 1 hour, more preferably at least 1.5 hours, most preferably at least 2 hours, or at least 2.5 hours, more preferably at least 3.5 hours, more preferably at least 4.5 hours.

Embodiment 1: A hemodynamic peptide, or a functional analog thereof, for use in the treatment of a human subject, the use comprising modifying hemodynamics in the human subject.

Embodiment 2: A hemodynamic peptide, or a functional analog thereof, for use in the treatment of a human subject considered at risk or suffering from fluid overload, the use comprising modifying hemodynamics in the human subject.

Embodiment 3: A hemodynamic peptide, or a functional analog thereof, for use in the treatment of a human subject considered at risk or suffering from excess vasopressor/inotropic use, the use comprising modifying hemodynamics in the human subject.

Embodiment 4: A hemodynamic peptide, or a functional analog thereof, for use in the treatment of a human subject, wherein the human subject is subjected to induced trauma and wherein the use comprises modifying hemodynamics in the human subject.

Embodiment 5: A hemodynamic peptide, or a functional analog thereof, for use in the treatment of a human subject having impaired kidney function, the use comprising modifying hemodynamics in the human subject.

Embodiment 6: A hemodynamic peptide, or a functional analog thereof, for use as in accordance with any one of embodiments 1-5, wherein the use reduces fluid retention in the human subject.

Embodiment 7: A hemodynamic peptide, or a functional analog thereof, for use in accordance with any one of embodiments 1-6 wherein the use comprises a reduced use of vasopressive agents.

Embodiment 8: A hemodynamic peptide, or a functional analog thereof, for use in accordance with any one of embodiments 1-7 wherein the use comprises a reduced fluid intake.

Embodiment 9: A hemodynamic peptide, or a functional analog thereof, for use in accordance with embodiment 7, wherein the reduced use of vasopressive agents comprises a reduced duration of vasopressive agent use.

Embodiment 10: A hemodynamic peptide, or a functional analog thereof, for use in accordance with any one of embodiments 6-9, wherein the subject is subjected to induced trauma.

Embodiment 11: A hemodynamic peptide, or a functional analog thereof, for use in accordance with any one of embodiments 6-10 wherein the use improves kidney function in the human subject.

Embodiment 12: A hemodynamic peptide, or a functional analog thereof, for use in accordance with embodiment 11, wherein the improved kidney function involves an improved GFR rate.

Embodiment 13: A hemodynamic peptide, or a functional analog thereof, for use in accordance with any one of embodiments 6-12, wherein the human subject has impaired kidney function the impaired kidney function being AKI.

Embodiment 14: A hemodynamic peptide, or a functional analog thereof, for use as in accordance with any one of embodiments 1-13, wherein the use reduces leakage of plasma from the blood to peripheral tissue and/or organs.

Embodiment 15: A hemodynamic peptide, or a functional analog thereof, for use in accordance with any one of embodiments 1-14, wherein the use is in a human subject suffering from or at risk of heart failure.

Embodiment 16: A hemodynamic peptide, or a functional analog thereof, for use in accordance with any one of embodiments 1-15, wherein the use is in a human subject at risk of having edema.

Embodiment 17: A hemodynamic peptide, or a functional analog thereof, for use in accordance with any one of embodiments 4-16, wherein the human subject has been subjected to induced trauma, the induced trauma being surgery.

Embodiment 18: A hemodynamic peptide, or a functional analog thereof, for use in accordance with embodiment 17, wherein the surgery requires a cardiopulmonary bypass.

Embodiment 19: A hemodynamic peptide, or a functional analog thereof, for use in accordance with any one of embodiments 1-18, wherein the peptide is administered into the bloodstream.

Embodiment 20: A hemodynamic peptide, or a functional analog thereof, for use in accordance with embodiment 19, wherein the peptide is administered at a rate of at least 70 mg/kg body weight/hour.

Embodiment 21: A hemodynamic peptide, a functional analog thereof, for use in accordance with embodiment 19 or embodiment 20, wherein the peptide is administered for at least 1 hour.

Embodiment 22: A hemodynamic peptide, a functional analog thereof, for use in accordance with any one of embodiments 17-21, wherein the administration is during surgery.

Embodiment 23: A hemodynamic peptide, or a functional analog thereof, for use in accordance with any one of embodiments 1-22, wherein the administration is during anti-cancer treatment.

Embodiment 24: A hemodynamic peptide, or a functional analog thereof, for use in accordance with any one of embodiments 1-23, wherein the administration is during an adverse drug reaction.

Embodiment 25: A hemodynamic peptide, or a functional analog thereof, for use in accordance with any one of embodiments 1-24, wherein the human subject is admitted into intensive care, and wherein the use improves parameters measured of the human subject, the parameters of the human subject determined to assess remaining in intensive care.

Embodiment 26: A hemodynamic peptide, or a functional analog thereof, for use in accordance with embodiment 25, wherein the improvement in parameters results in a reduced length of stay at intensive care.

Embodiment 27: A hemodynamic peptide, or a functional analog thereof, for use as in accordance with any one of embodiments 1-26, wherein the uses induces vasoconstriction.

Embodiment 28: A method of treatment comprising administering a hemodynamic peptide, or a functional analog thereof, to a human subject, the human subject being in need of maintaining hemodynamic stability.

Embodiment 29: A method of treatment comprising administering a hemodynamic peptide, or a functional analog thereof, to a human subject, the human subject being in need of improving hemodynamic stability.

Embodiment 30: A method of treatment comprising administering a hemodynamic peptide, or a functional analog thereof, to a human subject, the human subject having impaired kidney function, wherein the treatment of administering a hemodynamic peptide comprises maintaining or improving hemodynamic stability in the human subject.

Embodiment 31: A method of treatment comprising administering a hemodynamic peptide, or a functional analog thereof, to a human subject, the human subject being in need of improving an adverse drug reaction.

Embodiment 32: A method of treatment comprising administering a hemodynamic peptide, or a functional analog thereof, to a human subject, the human subject having or suspected of having Clarkson's disease (CLS).

Embodiment 33: A method of treatment comprising administering a hemodynamic peptide, or a functional analog thereof, to a human subject, the human subject having or suspected of having an adverse drug reaction affecting capillary leakage.

Embodiment 34: A method of treatment comprising administering a hemodynamic peptide, or a functional analog thereof, to a human subject, the human subject being in need of maintaining hemodynamic stability.

Embodiment 35: A method of treatment comprising administering a hemodynamic peptide, or a functional analog thereof, to a human subject, the human subject being in need of improving hemodynamic stability.

Embodiment 36: A method of treatment comprising administering a hemodynamic peptide, or a functional analog thereof, to a human subject, the human subject having impaired kidney function, wherein the treatment of administering a hemodynamic peptide comprises maintaining or improving hemodynamic stability in the human subject.

### Examples

A Phase 2 clinical trial was designed to test EA-230 (tetrapeptide AQGV; SEQ ID NO:1) in cardiac surgery (CABG) patients who frequently develop hemodynamic imbalances leading to organ dysfunction. The study was finalized in 2019 and results demonstrate significantly improved renal function after treatment with EA-230, with a statistically highly significant reduction in LOS (length of stay in ICU and Hospital) and a reduction of hospital re-admission. These beneficial effects of EA 230 translate into a significant better recovery of open-heart-surgery patients treated with EA 230, providing a distinct economic benefit. This positive outcome likely emerged from the during this Phase 2 trial unexpectedly shown and greatly beneficial effects of EA-230 on hemodynamic stability of intensive care patients that leads to a reduction in fluid overload, reduction of vasopressor use, and improvement in kidney functionality.

During cardiac surgery, 180 patients received (double-blinded, placebo-controlled, randomized) 90 mg/kg/hour EA-230 or placebo. The primary endpoint was safety. Efficacy was assessed by immunomodulation (plasma interleukin (IL)-6 concentrations), renal function (glomerular filtration rate using iohexol and creatinine [GFRiohexol, eGFRMDRD] and the incidence of acute kidney injury [AKI, RIFLE criteria]), cardiovascular effects (fluid balance, vasoactive agents) and general outcome (length-of-stay).

Median [IQR] age was 68 [62-74] years, 158/180 males. No safety concerns emerged. EA-230 did not modulate IL-6 (area under the curve 2730 [1968-3760] vs. 2680 [2090-3570] pg/ml*hour for EA-230 and placebo group respectively, p=0.80). GFR increased following surgery (meanΔ±SEM GFRiohexol 19±2 vs. 16±2 ml/min/1.73 m2 respectively, p=0.13, eGFRMDRD 6±1 vs. 2±1 ml/min/1.73 m2, respectively, p=0.01). EA-230 tended to prevent AKI (stage Injury: 7% vs. 18%, respectively, p=0.07). Patients in the EA-230 group needed less fluids compared to placebo-treated patients (217±108 vs. 605±103 ml, respectively, p=0.01), while the use of vasoactive agents was similar in both groups (p=0.39). Hospital length-of-stay was shorter in EA-230 treated patients (8 [7-11] vs. 10 [8-12] days, respectively, p=0.001).

Safety of EA-230

Final analysis of the EASI-study showed an excellent safety profile of treatment with EA-230. A continuous infusion of 90 mg/kg/hour EA-230 for up to 4 hours was well tolerated by patients undergoing elective CABG surgery. Pharmacokinetic studies indicate that EA-230 is rapidly (within 5-10 minutes) cleared from the circulation when infusion is terminated. Patients that received EA-230 seemed to experience less (serious) adverse events and less mayor clinical adverse events. In conclusion, the safety profile of EA-230 in patients undergoing elective CABG surgery was comparable if not better to the safety profile of patients receiving a continuous placebo infusion.

Efficacy of EA-230

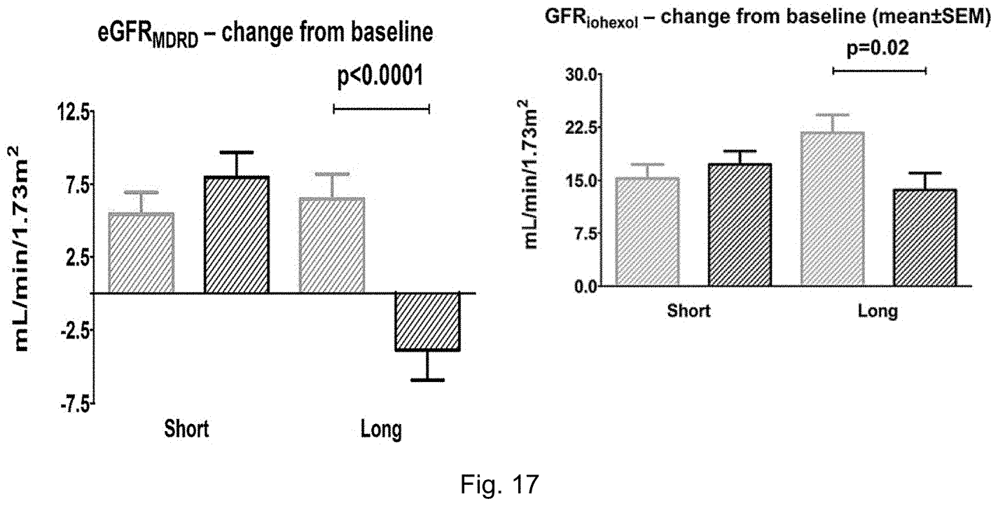

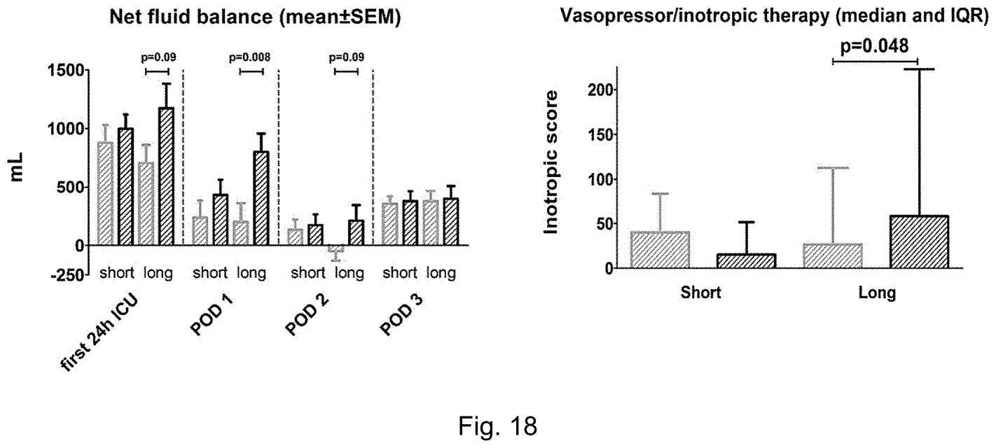

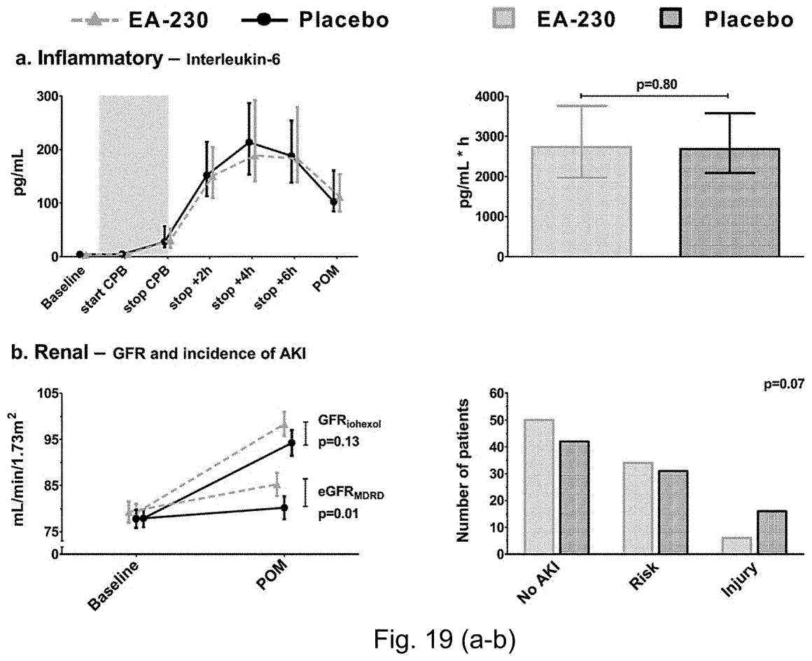

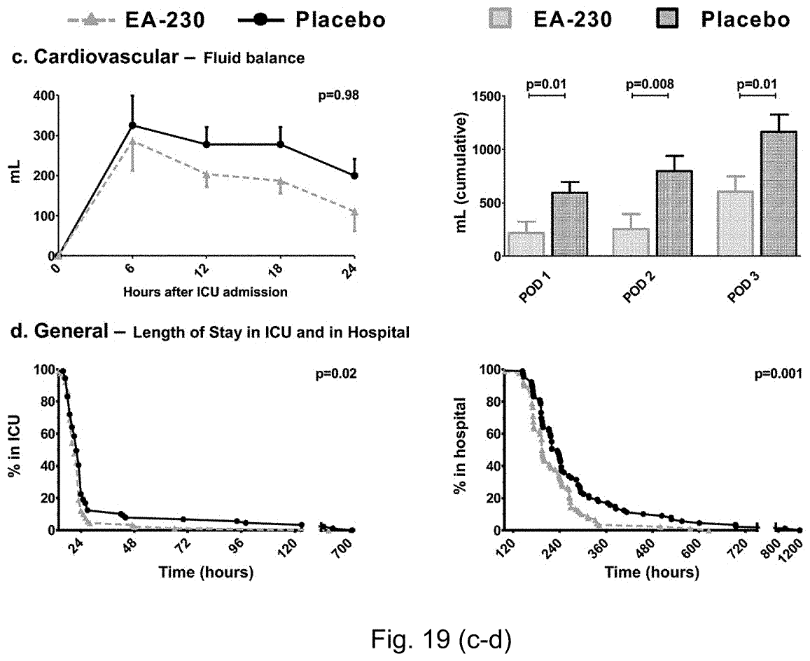

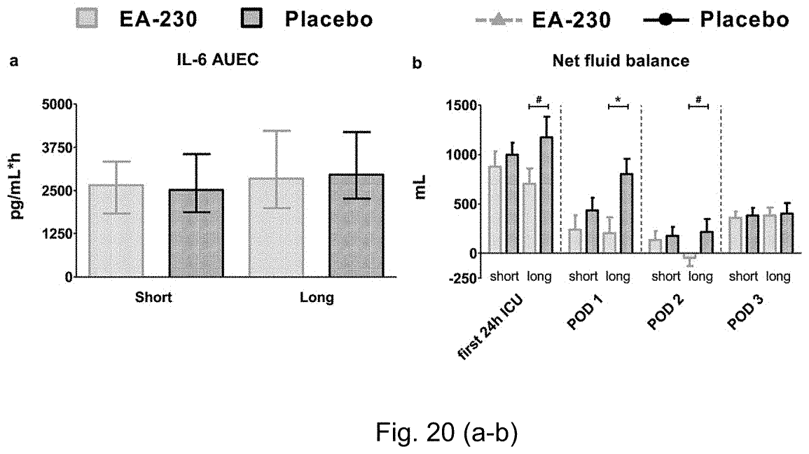

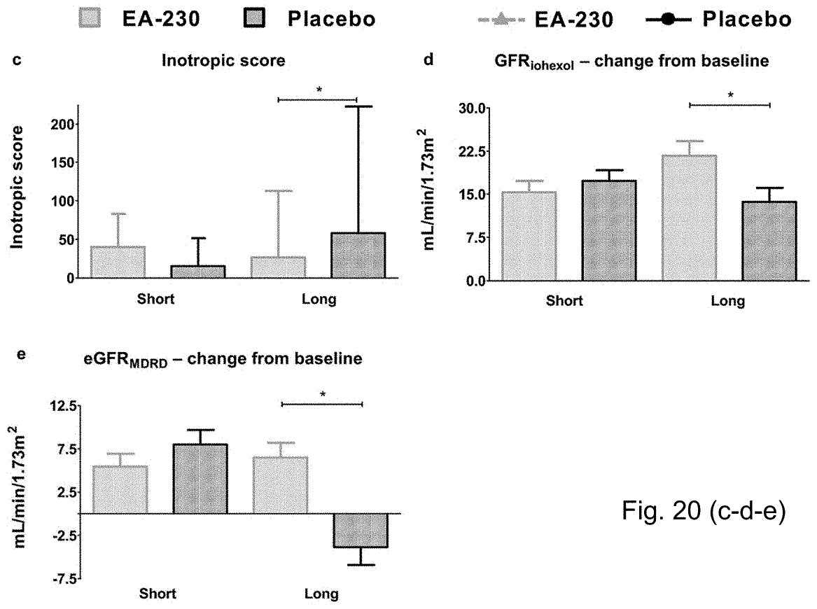

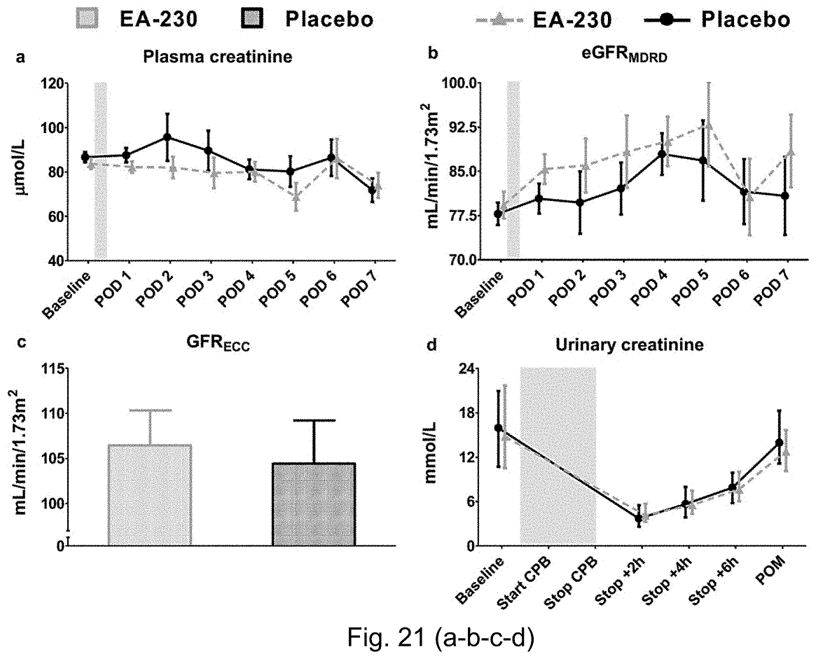

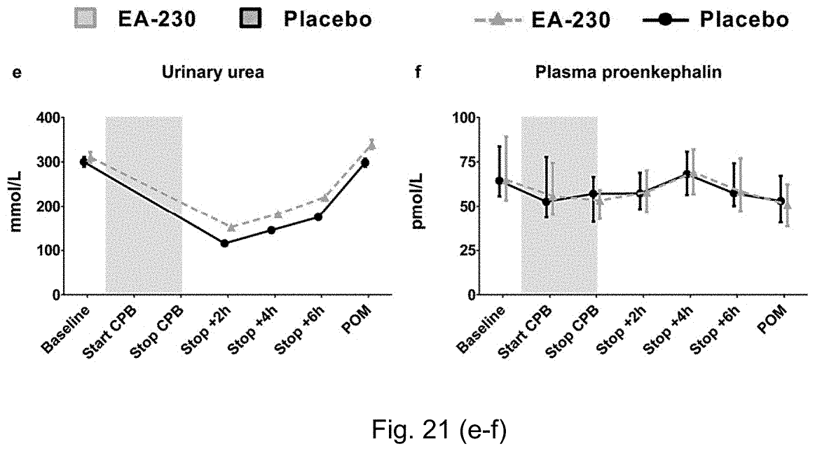

Continuous infusion of 90 mg/kg/hour EA-230 for up to 4 hours in patients undergoing elective CABG surgery demonstrated unexpected but distinct clinical benefits of treatment with EA-230. Foremost, treatment with EA-230 (n=90) resulted in a highly significant reduction of about 3 days of total post-operative length-of-stay. While no effects on CABG-induced cytokine responses (with primary endpoint IL-6) were found, treatment with EA-230 distinctly caused an overall significant improvement of hemodynamic stability, based on reduced need of blood pressure (vasopressor/inotropic) medication and reduced need for fluid therapy, therewith preventing against post-operative fluid overload. Also, a significant overall improvement of kidney function was found. As detailed herein, prolonged treatment with EA-230 during surgery provides increased clinical benefits of patient recovery rates after surgery. The results also show beneficial effects of prolonged treatment (long=longer than median) with EA-230 on kidney function, measured as glomerular filtration rate (GFR), as compared with patients that were treated shorter than median treatment. The findings (FIG. 17) indicate highly significant statistical improvements of kidney function after surgery due to prolonged treatment of patients with EA-230 during elective CABG surgery. Similarly, post-operative hemodynamic stability (FIG. 18, measured as need for vasopressor/inotropic and/or fluid therapy) after surgery, benefits significantly from prolonged use of EA-230 during surgery.

Summary of EA-230 Effects

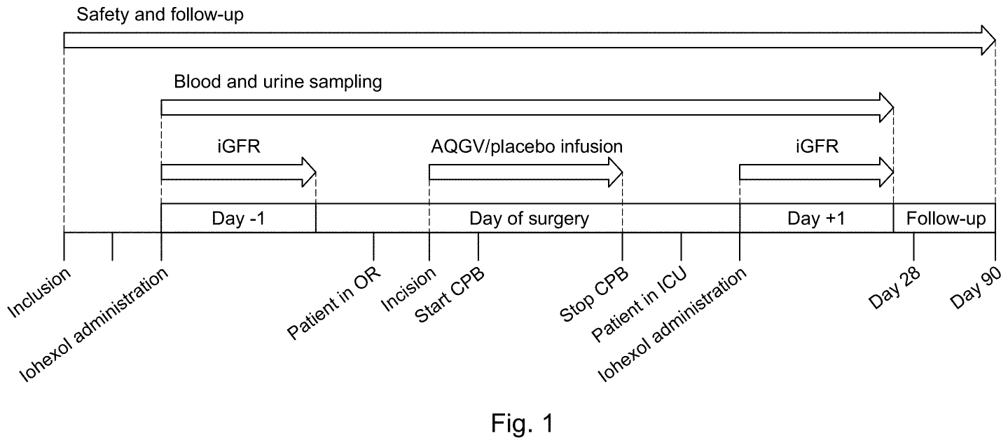

Early administration led us to the detection of novel and highly beneficial effects of EA-230 on hemodynamics, kidney function, length of stay in ICU and hospital, which relate to improved hemodynamic stability. Treatment of patients with EA-230 during surgery significantly reduced the need for hemodynamic therapy (combined fluid therapy and blood pressure medication) after surgery (p=0.006). Besides these improved hemodynamics, EA-230 significantly improved kidney function (as determined by its effects on the glomerular filtration rate) and plasma levels of kidney function biomarker creatinine (p=0.003). It also significantly shortened post-surgery recovery stay at the ICU and significantly reduced length of stay in the hospital (FIG. 1). On average, EA-230-treated patients needed about 8 days of hospital care where placebo-treated patients needed about 10 days. Also, fewer EA-230-treated patients needed re-hospitalization than placebo-treated patients did.

Effects of EA-230 in Human Patients

A prospective, randomized, double-blind, placebo-controlled study was performed in which 180 elective patients, undergoing on-pump coronary artery bypass grafting, with or without concomitant valve surgery, were enrolled. Patients were randomized in a 1:1 ratio and received either EA-230, 90 mg/kg/hour, or a placebo. These were infused at the start of the surgical procedure until the end of the use of the cardiopulmonary bypass. The main focus in this first-in-patient study was on safety and tolerability of EA-230. The primary efficacy endpoint was the modulation of the inflammatory response by EA-230 quantified as the change in interleukin-6 plasma concentrations after surgery. A key secondary endpoint was the effect of EA-230 on renal function.

Design and Setting

The present study was a single-center, prospective, double-blind, placebo-controlled, randomized, single-dose phase II study. It has an adaptive design to evaluate the safety and immunomodulatory effects of EA-230 in patients undergoing on-pump cardiac surgery for coronary artery bypass grafting (CABG) with or without concomitant valve surgery. 180 eligible patients were included and were randomized to receive either active or placebo treatment in a 1:1 ratio. This was a first-in-patient safety and tolerability study, of which the primary efficacy objective was to assess the immunomodulatory effects of EA-230. The key-secondary efficacy endpoint was the effect of EA-230 on renal function. This study was described in accordance with the Standard Protocol Items: Recommendations for Interventional Trial (SPIRIT) guidelines, and registered at clinicaltrials.gov under number NCT03145220.

Randomization and Stratification

On the morning of surgery, patients were randomized by non-blinded independent study personnel for active or placebo treatment. Study personnel used Good-Clinical-Practice-approved data management software (Castor EDC, Amsterdam, The Netherlands) in this process. The Castor system applies a stratified randomization to ensure equal distribution between active and placebo treatment of patients with known risk factors for adverse outcomes. Three strata were included: 1) a CABG procedure with or without concomitant valve surgery; 2) pre-operative renal function with an estimated GFR of ≤30, 31-90 and >90 ml/min/1.73 m2; and 3) a EuroSCORE II of <4 or ≥4 (Nashef et al., Eur. J. Cardiothora. Surg. 2012 April; 41(4):734-44).

Blinding

Double-blind conditions were maintained for all patients, the attending physicians and the medical study team personnel involved in all blinded study procedures, data collection and/or data analyses. Non-blinded study personnel not involved in any other study procedures prepared the study medication. Infusion systems and solutions for active and placebo treatment were identical in appearance and texture. Unblinding was authorized by the sponsor after completion of the study, performance of a blinded data review and locking of the database.

Study Intervention

Intravenous infusion of EA-230, 90 mg/kg/hour, or placebo, was initiated at the moment of first surgical incision using an automated infusion pump. Infusion rate was set at 250 ml/hour, and infusion was continued until cessation of the CPB, or after 4 hours of continuous infusion, whichever comes first.

The EA-230 formulation was packed in sterile 5 ml glass vials, containing 1500 mg/vial, dissolved in water for injection at a final concentration of 300 mg/ml with an osmolality of 800 to 1000 mOsm/kg. The placebo formulation consisted of sodium chloride diluted in water for injection in identical sterile 5 ml glass vials containing 29 mg/ml to reach a solution with an identical osmolality. EA-230 and placebo were prepared for continuous intravenous infusion with an osmolality of <400 mOsm/Kg by adding the appropriate amount of EA-230 or placebo to 1000 ml normal saline under aseptic conditions. Placebo and active treatment vials, were manufactured by HALIX BV (Leiden, the Netherlands).

Adverse Events (AEs)

All AEs were judged by the investigators with regard to severity (“mild, moderate, or severe”) according to Common Terminology Criteria for Adverse Events guidelines 4.030 and their perceived relation to the study drug (“definitely, probably, possibly, or unrelated/unlikely to be related”). SAEs or SUSARs include death, life-threatening disease, persistent and/or significant disability and/or incapacity, and hospitalization and/or prolongation of inpatient hospitalization.

Ethical Considerations, Data Quality Assurance & Patient and Public Involvement.

The study was conducted in accordance with the ethical principles of the Declaration of Helsinki (ICH E6(R1)), the Medical Research Involving Human Subjects Act, guidelines of Good Clinical Practice and European Directive (2001/20/CE). Informed consent was obtained before any study-specific procedures were performed. Data was handled confidentially and anonymously and Good-Clinical-Practice standards were applied. The handling of patient data in this study complies with the Dutch Personal Data Protection Act (in Dutch: Wet Bescherming Persoonsgegevens, WBP). Patients and the public were not involved in the design and/or the conduct of the study protocol. Study outcome was disseminated to all study participants individually. The burden of the intervention was assessed by the independent ethics committees CMO and CCMO, which include lay members.

Results

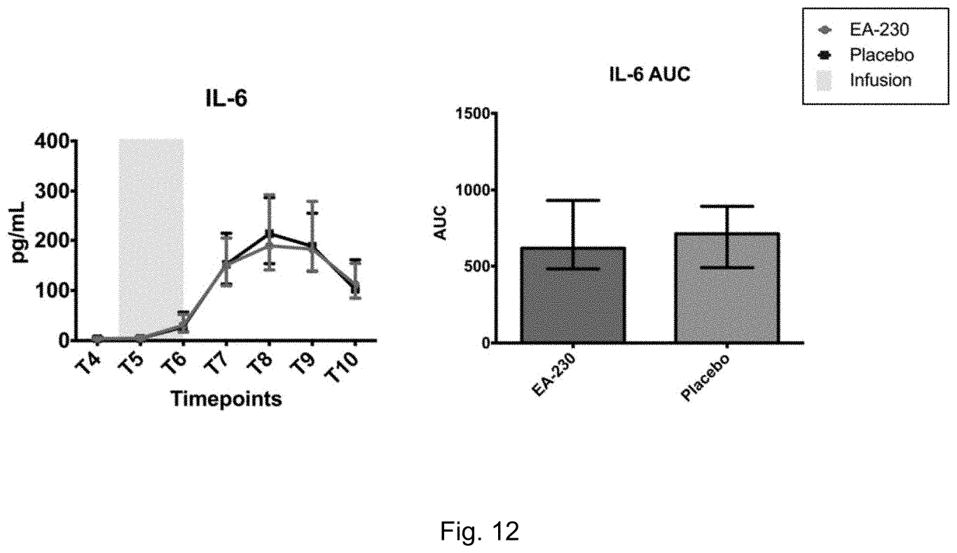

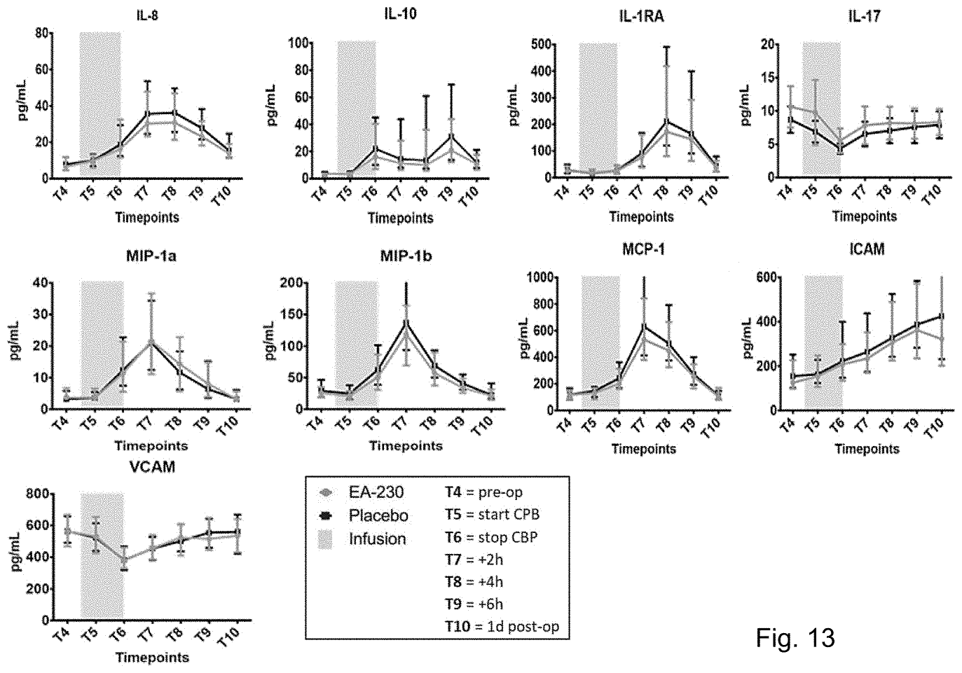

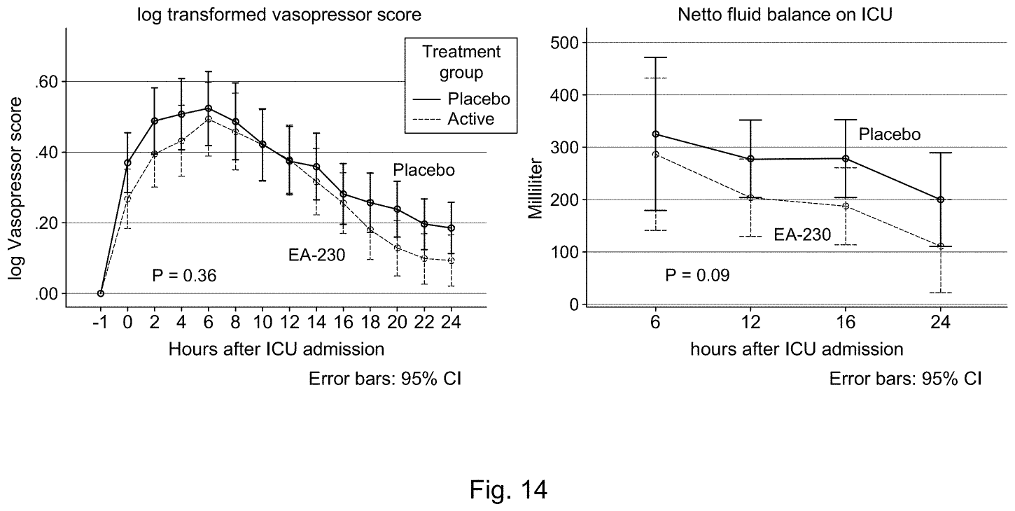

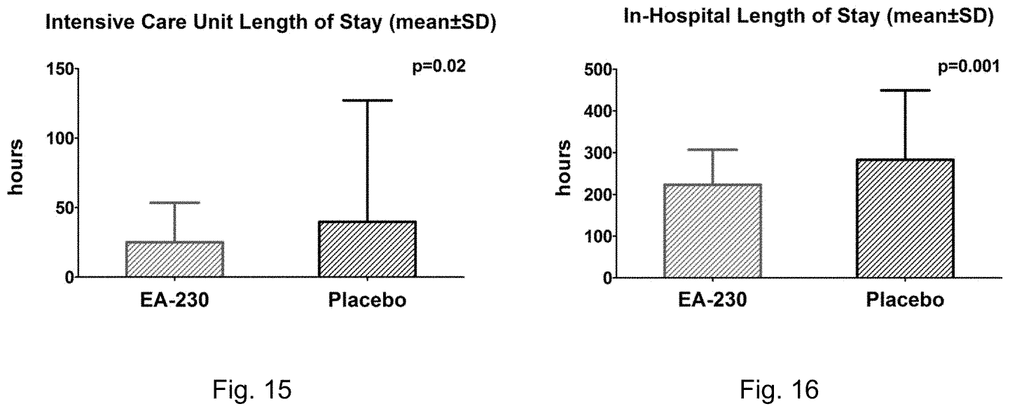

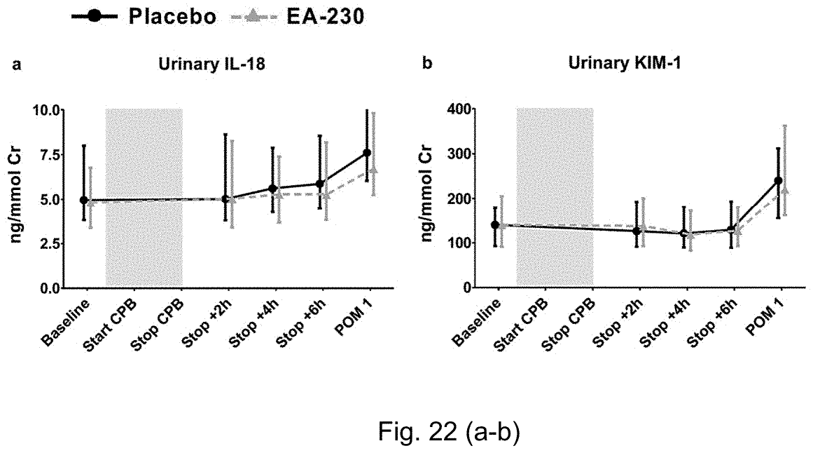

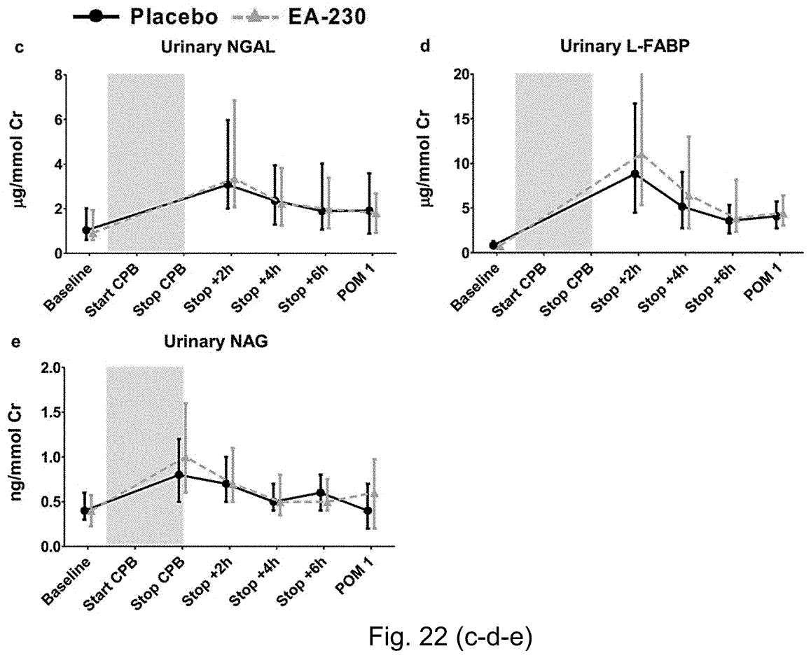

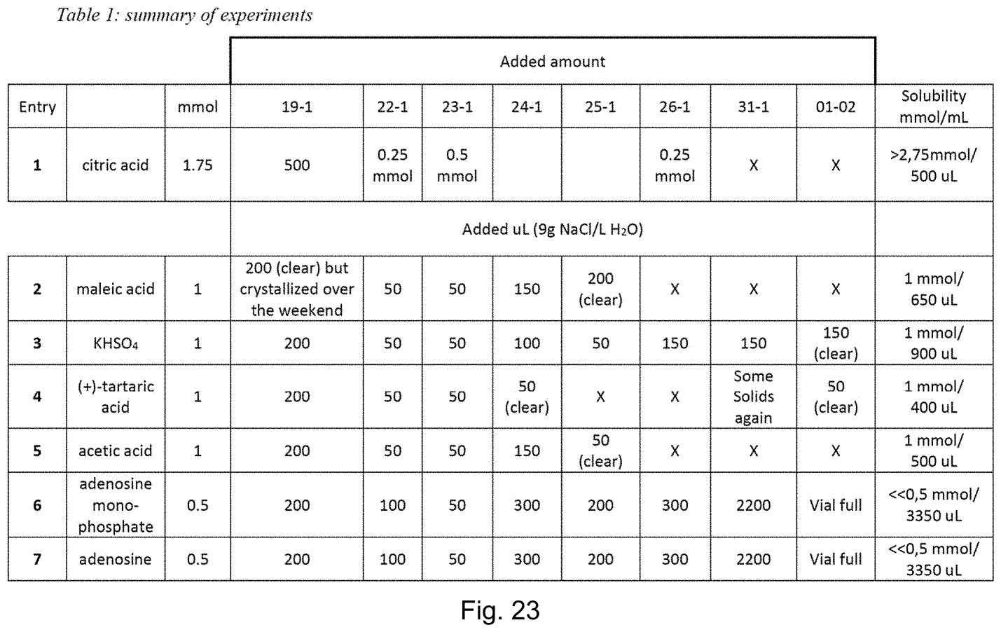

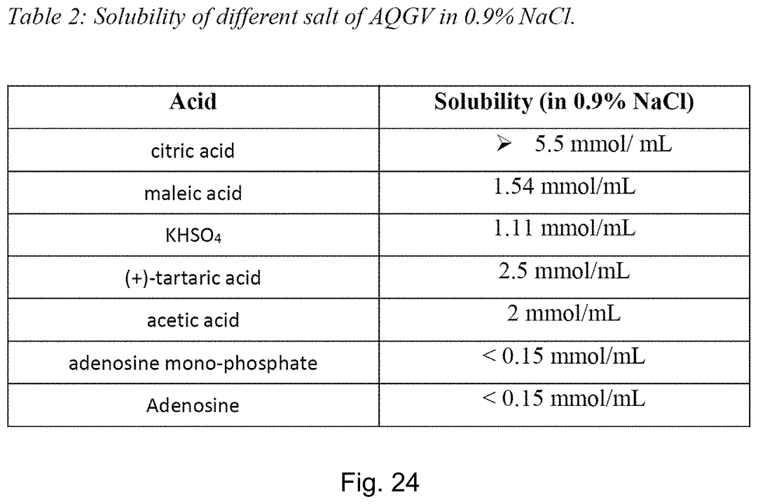

When assessing the data obtained during the clinical trial, strikingly, no immunomodulatory or anti-inflammatory effects were apparently observed as no significant difference of plasma levels between the EA-230 and placebo group were observed for IL-8, IL-10, IL-1RA, IL-17, MCP-1 and ICAM and other cytokines tested. This was also the case for IL-6 plasma levels, the primary endpoint of the study (see FIG. 12) and for inflammatory renal injury markers IL-18, KIM1, NGAL, L-FABP and NAG (see FIG. 22). Strikingly, significantly less patients suffering from fluid retention were found the EA-230 treatment group (see table 1). Various parameters were further analyzed and it was found that hemodynamic parameters (such as vasopressor use and/or fluid balance) and/or kidney parameters were advantageously affected by the use of EA-230 as compared with placebo.

TABLE 1
Adverse events (AEs) in the EASI-study. AEs, serious adverse events (SAE), and suspected
unexpected serious adverse reaction (SUSAR) with differences between treatment groups
are listed here. Significantly less (Chi Square P < 0.05) AEs were found in the
EA-230 treatment group (217) than in the placebo treated group (283). Significantly
less patients (Chi Square P < 0.05) suffering from fluid retention were found
the EA-230 treatment group (n = 2) than in the placebo treated group (n = 11), p < 0.05.

| | EA-230 (N = 91) | | | Placebo (N = 89) | | | Overall (N = 180) | | |
|---|---|---|---|---|---|---|---|---|---|
| | e | n | (%) | e | n | (%) | e | n | (%) |
| Any AE | 217 | 78 | (85.7) | 283 | 81 | (91) | 500 | 159 | (88.3) |
| Any SAE | 23 | 12 | (13.2) | 19 | 17 | (19.1) | 32 | 29 | (16.1) |
| Any SUSAR | 0 | 0 | (0) | 1 | 1 | (1.1) | 1 | 1 | (0.6) |
| AE of mild intensity | 188 | 76 | (83.5) | 231 | 78 | (87.6) | 419 | 154 | (85.6) |
| AE of moderate intensity | 23 | 22 | (24.2) | 45 | 27 | (30.3) | 68 | 49 | (27.2) |
| AE of severe intensity | 6 | 5 | (5.5) | 7 | 4 | (4.5) | 13 | 9 | (5) |
| Blood and lymphatic | | | | | | | | | |
| system disorders | | | | | | | | | |
| Overall | 6 | 5 | (5.5) | 8 | 8 | (9) | 14 | 13 | (7.2) |
| Anemia | 5 | 5 | (5.5) | 8 | 8 | (9) | 13 | 13 | (7.2) |
| Hemorrhagic diathesis | 1 | 1 | (1.1) | 0 | 0 | (0) | 1 | 1 | (0.6) |
| Gastrointestinal disorders | | | | | | | | | |
| Overall | 27 | 21 | (23.1) | 36 | 23 | (25.8) | 63 | 44 | (24.4) |
| Nausea | 15 | 15 | (16.5) | 12 | 12 | (13.5) | 27 | 27 | (15) |
| Infections and infestations | | | | | | | | | |
| Overall | 15 | 13 | (14.3) | 22 | 17 | (19.1) | 37 | 30 | (16.7) |
| Metabolism and nutrition | | | | | | | | | |
| disorders | | | | | | | | | |
| Overall | 10 | 10 | (11) | 22 | 14 | (15.7) | 32 | 24 | (13.3) |
| Fluid retention | 2 | 2 | (2.2) | 9 | 9 | (10.1) | 11 | 11 | (6.1) |
| Psychiatric disorders | | | | | | | | | |
| Overall | 9 | 9 | (9.9) | 14 | 13 | (14.6) | 23 | 22 | (12.2) |
| Delirium | 5 | 5 | (5.5) | 9 | 9 | (10.1) | 14 | 14 | (7.8) |
| Renal and urinary | | | | | | | | | |
| disorders | | | | | | | | | |
| Overall | 4 | 4 | (4.4) | 12 | 11 | (12.4) | 16 | 15 | (8.3) |

N = Number of patients involved
n = Number of patients experiencing the event
e = Number of events

TABLE 2
Average on-pump length of patients with average age of patients,
split in quartiles Q1, Q2, Q3 and Q4 of pump length, and thus
of treatment duration, and of all patients tested (Q1-Q4).

| | Treatment | Average on-pump-length | Age in years |
|---|---|---|---|
| Quartiles | group | in minutes (+/−SD) | (+/−SD) |
| Q1 | EA-230 | 112 +/− 12 | 68.5 +/− 7.3 |
| Q1 | Placebo | 113 +/− 7 | 70.3 +/− 7.9 |
| Q2 | EA-230 | 137 +/− 5 | 66.5 +/− 9.5 |
| Q2 | Placebo | 136 +/− 6 | 68.1 +/− 6.9 |
| Q3 | EA-230 | 164 +/− 8 | 66.3 +/− 8.7 |
| Q3 | Placebo | 159 +/− 8 | 68.3 +/− 11.0 |
| Q4 | EA-230 | 211 +/− 24 | 65.0 +/− 7.4 |
| Q4 | Placebo | 207 +/− 22 | 64.0 +/− 10.6 |
| Q1-Q4 | EA-230 | 156 +/− 37 | 66.5 +/− 8.3 |
| Q1-Q4 | Placebo | 153 +/− 39 | 67.7 +/− 9.3 |

Hemodynamic Stability in the EASI-Study

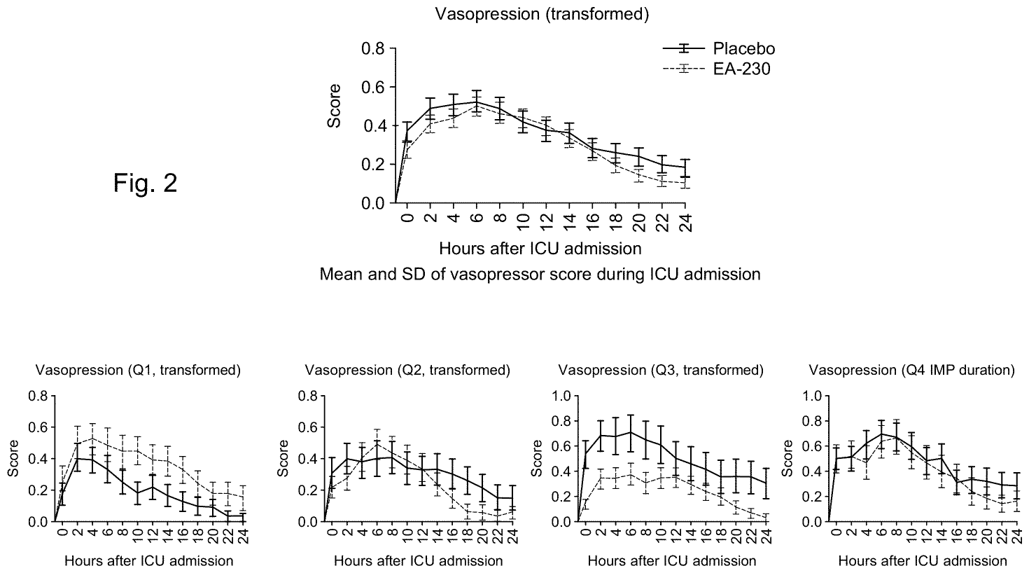

In FIG. 2 the use of vasopressors is shown. In general, the use of vasopressors was reduced in the group that was treated with EA-230. Patients were divided in quartiles based on treatment duration. In Table 3, descriptive frequencies of the 2 variables: days on vasopressin and netto fluid balance day 0-2 (first 72 hours) are shown. The groups were split in patients without acute kidney injury (AKI) and with AKI, as well in patients without treatment (placebo) and with treatment with EA-230 (active). EA-230 decreased the net (netto) fluid balance in patients both with and without AKI. EA-230 decreased the need for vasopressors in patients with AKI.

TABLE 3
Frequencies
Statistics

| RIFLE | | | | | Netto |
|---|---|---|---|---|---|
| score | | | | | fluid |
| (incl urine | | | | days on | balance |
| output) | Treatment group | | | vasopressin | day 0-2 |
| no AKI | Placebo | N | Valid | 42 | 42 |
| | | | Missing | 0 | 0 |
| | | Mean | | 1.38 | 1951.97 |
| | | Median | | 1.00 | 1957.15 |
| | | Std. Deviation | | .795 | 1027.255 |
| | Active | N | Valid | 51 | 51 |
| | | | Missing | 0 | 0 |
| | | Mean | | 1.37 | 1348.47 |
| | | Median | | 1.00 | 1213.70 |
| | | Std. Deviation | | 1.131 | 1304.211 |
| AKI | Placebo | N | Valid | 47 | 47 |
| | | | Missing | 0 | 0 |
| | | Mean | | 2.23 | 3342.04 |
| | | Median | | 2.00 | 2965.20 |
| | | Std. Deviation | | 2.108 | 2275.286 |
| | Active | N | Valid | 39 | 39 |
| | | | Missing | 0 | 0 |
| | | Mean | | 1.97 | 2549.16 |
| | | Median | | 2.00 | 2328.80 |
| | | Std. Deviation | | 1.423 | 1517.623 |

Modulation of Fluid Balance and Vasopressor Use by Treatment with EA-230

The effects of EA-230 versus placebo were tested in uni- and multivariate models (see Table 4). Input/independent variable: treatment group (EA-230 or placebo). Output/dependent variables were: endpoint of fluid balance first 72 hours, days on vasopressin or vasopressor score (area under the curve). Effects of EA-230 versus placebo were tested on two combined variables in model A: fluid balance first 72 hours+days on vasopressin and model B: Fluid balance first 72 hours+vasopressor score AUC. The results of testing in both multivariate models showed significant improvement of hemodynamic parameters in patients receiving EA-230. This was observed in model A (fluid balance first 72 hours+days on vasopressin) p=0.006 and in model B (fluid balance first 72 hours+vasopressor score AUC) p=0.008. In the group of patients that showed no AKI, hemodynamic effects of EA-230 were significantly better as well, illustrating that improvement in hemodynamics can occur independent of kidney failure.

TABLE 4
Goal-directed hemodynamic therapy by EA-230. An analysis
is shown for model A for the total group and for subgroups
of acute kidney injury split conform the RIFLE criteria:
No AKI (placebo n = 42, EA-230 n = 50), Risk
(placebo n = 31, EA-230 n = 34), and Injury
(placebo n = 16, EA-230 n = 6). The corresponding p-values are listed.

| | | TREATMENT EFFECTS ON: | | |
|---|---|---|---|---|
| | | Univariate 1. | Univariate | Multivariate |
| Significant improvement of | | Fluid balance | 2. Days on | 1 and 2 |
| active over placebo | | first 72 hours | vasopressin | combined |
| RIFLE stage | Total group | 0.441 | 0.807 | 0.006 |
| | No AKI | 0.017 | 0.996 | 0.048 |
| | Risk | 0.807 | 0.564 | 0.753 |
| | Injury | 0.051 | 0.055 | 0.114 |

Uni- and multivariate general linear model analysis

Combined, these results indicate that the use of EA-230 can improve and/or maintain hemodynamics in human patients, as assessed by, among other things, affecting the duration of vasopressor use, amount of vasopressor administered and/or fluid balance. In particular, EA-230 improves hemodynamic stability after open heart surgery in humans. Permeability governs the amount of fluid leaking from blood vessels. Administration of fluid therapy generally increases leakage. Based on Phase II trial patient observations, a significant reduction of adverse fluid retention (fluid leakage with fluid overload) was found in patients treated with EA-230 (p=0.03). Also, contractility governs tone. It is often adjusted by administration of blood-pressure medications, which, however, may show major detrimental side effects. Based on Phase II trial patient observations, a considerable reduction of required blood pressure medication use was found in the half of patients treated longest with EA-230 (>156 min; p=0.093). Mean maximum concentrations (mean Cmax) were also found as determined in vivo in humans for EA-230 in the Phase II clinical trial. Mean arterial Cmax found: 30500 ng/ml (range 12500 to 57500 ng/ml). Mean venous Cmax found: 68400 ng/ml (range 19600 to 113000 ng/ml)

EA-230 has an Advantageous Effect on Kidney Function

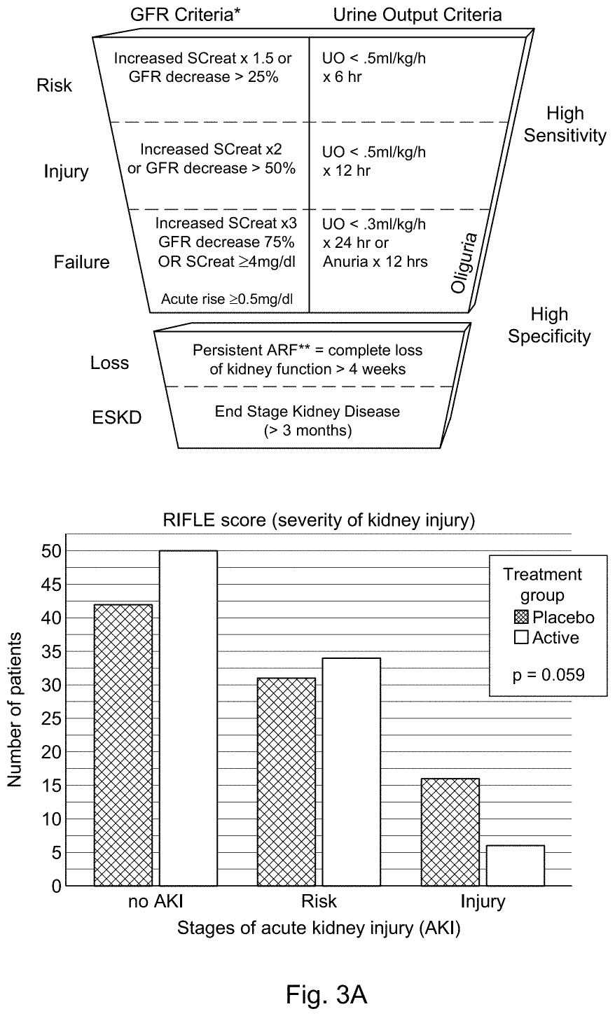

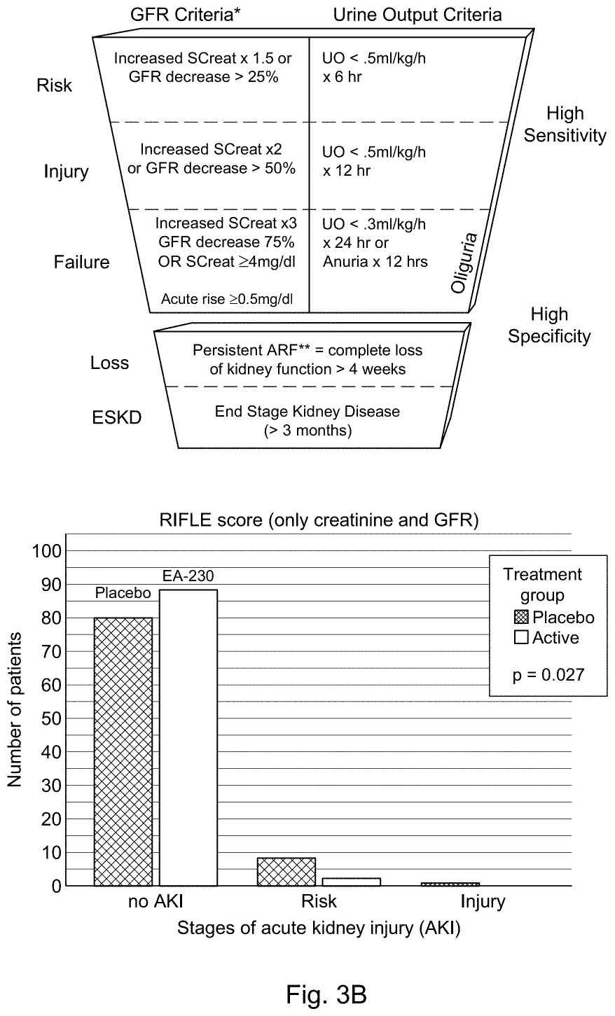

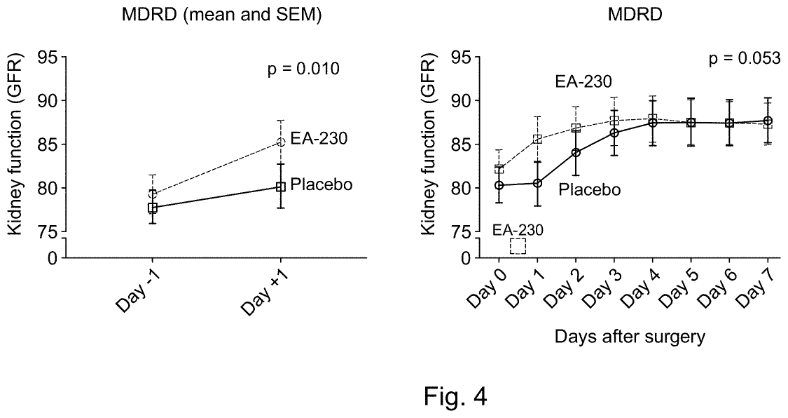

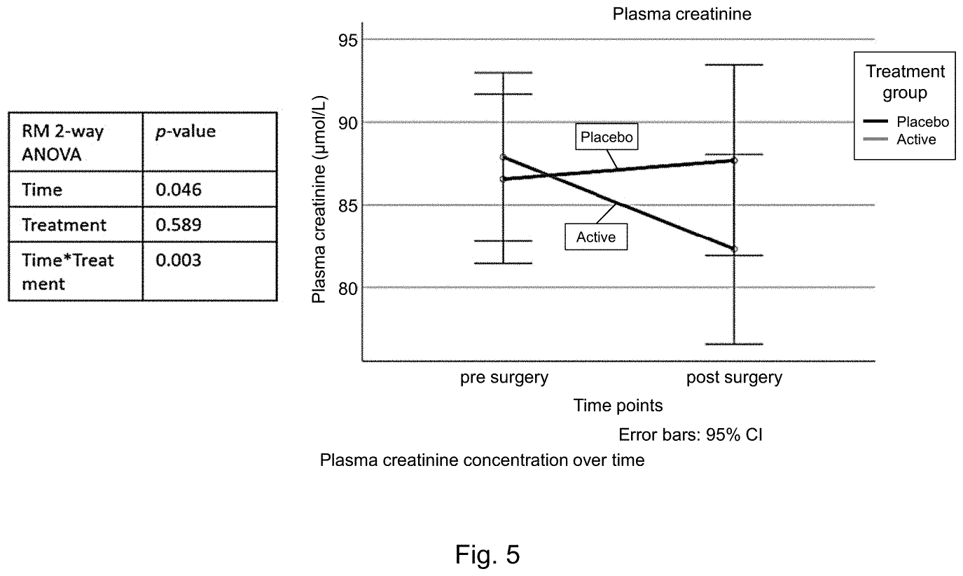

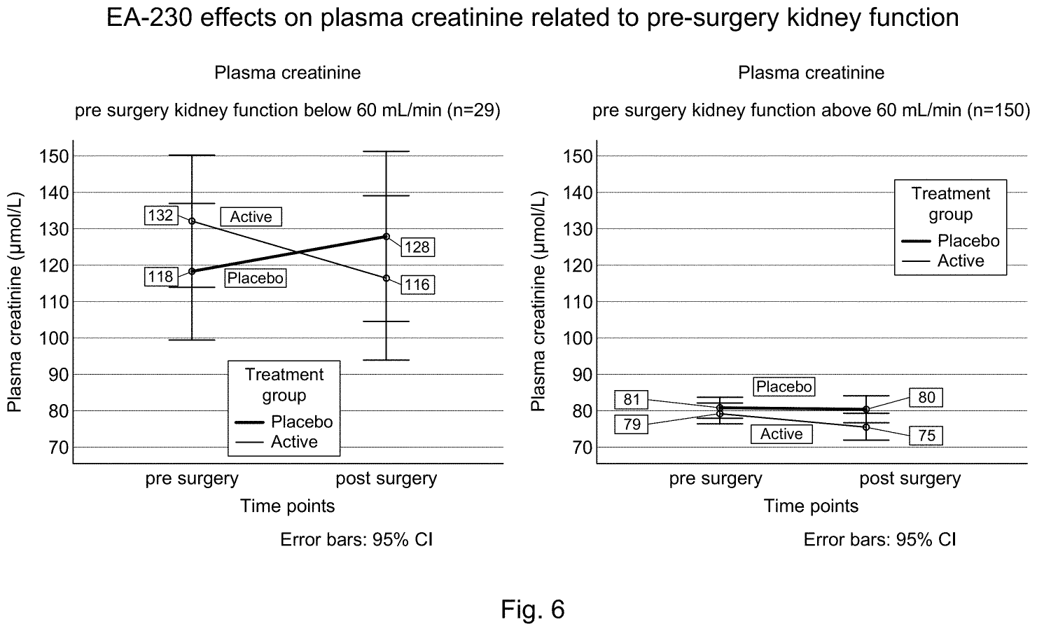

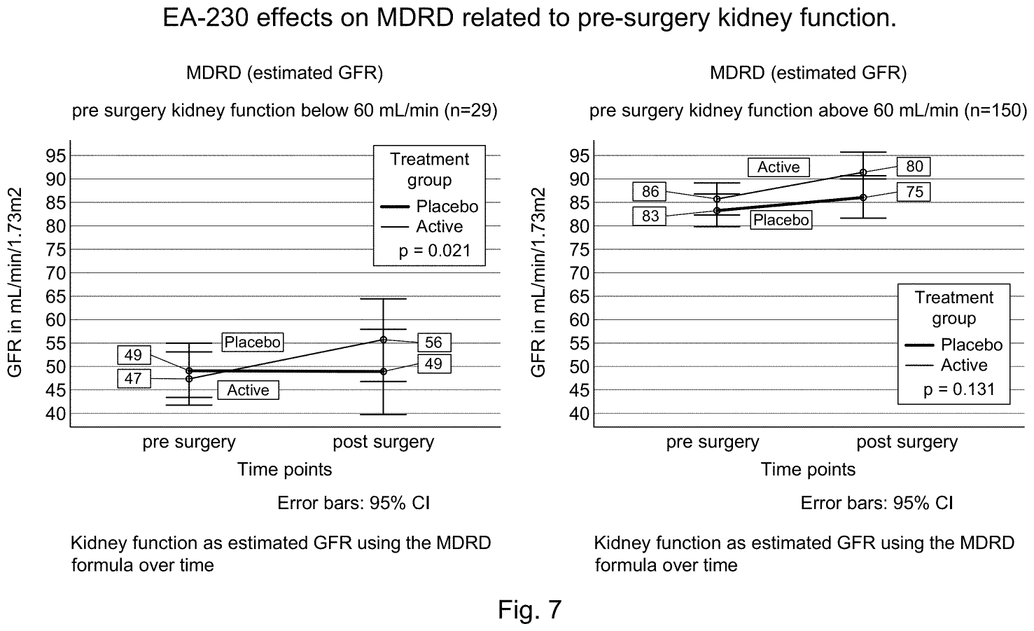

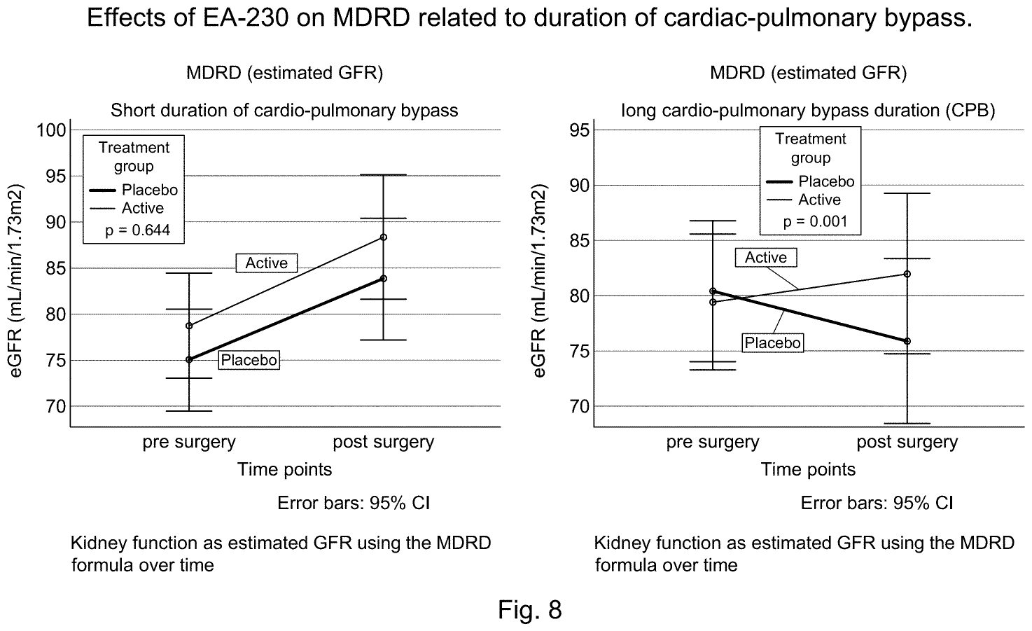

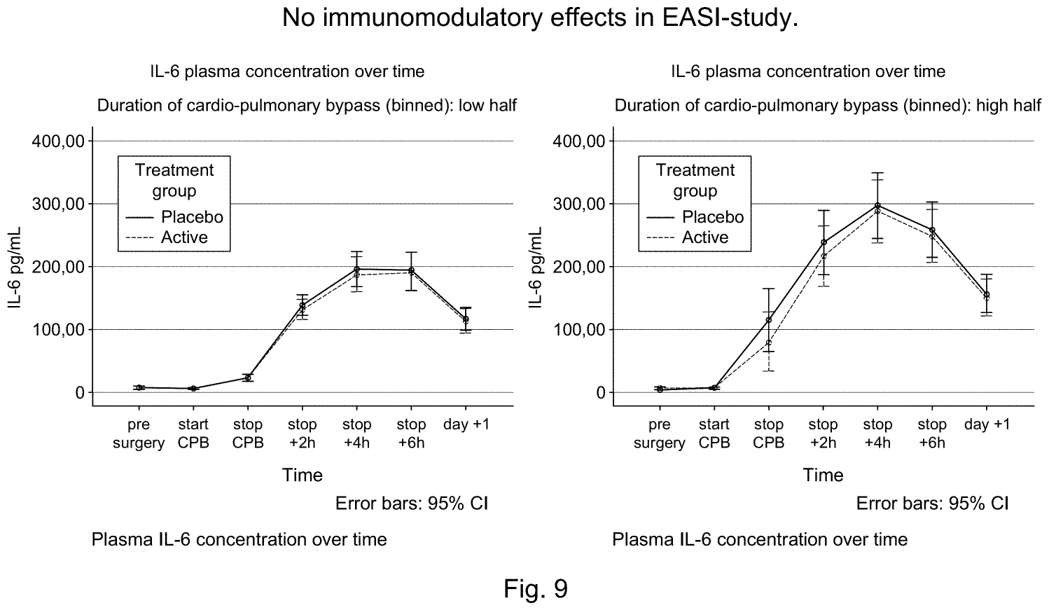

Effects of EA-230 on modulation in incidence of different stages of acute kidney injury (AKI) were determined according to the RIFLE criteria (RIFLE: risk, injury, failure, loss of kidney function, and end-stage kidney disease classification, Clin. Kidney J. 2013 February; 6(1): 8-14). In the EA-230 group, the number of patients with no AKI increased, whereas the number of patients in the Injury category of the RIFLE criteria decreased (see FIG. 3). Furthermore, the use of EA-230 significantly improved GFR after surgery (FIG. 4). Creatinine clearance, a biomarker of kidney function, was significantly improved post-surgery in patients treated with EA-230 (FIG. 5). When pre-surgery kidney function was taken into account, clearance of creatinine was significantly improved when EA-230 was used, when pre-surgery kidney function was below 60 ml/min (FIG. 6, left). When pre-surgery kidney function was above 60 ml/min, no differences were observed (FIG. 6, right). Similar observations were made based on the GFR parameter (FIG. 7). Treatment with EA 230 significantly improved estimated GFR after surgery when compared with estimated GFR before surgery, where treatment with placebo did not. When kidney function was above 60 ml/min/1.73 m2, no differences were found between groups. Also, when patients had a long duration of cardio-pulmonary bypass, treatment with EA-230 significantly improved GFR after surgery when compared with GFR before surgery (FIG. 8; treatment long>156 min; p=0.001). Combined, these results indicate that the use of EA-230 can improve and/or maintain kidney function in human patients.

Length of Stay in ICU, Hospital and Readmissions

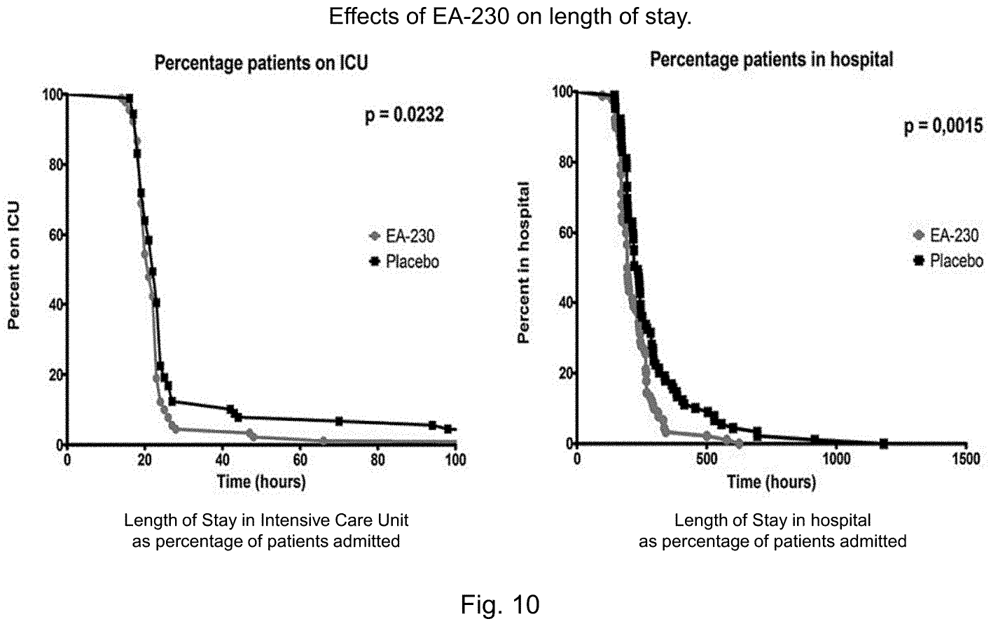

In the study, effects on length of stay at the ICU of patients and length of stay in the hospital (inpatient care) were investigated (see FIG. 10). Treatment with EA-230 resulted in a significant reduction of the length of stay (LOS) at the ICU as well as at the hospital. LOS in the ICU and the hospital was reduced in the EA-230 group. The patients treated with EA-230 also showed a considerable (p=0.09) reduction of the number of re-admissions to the hospital up to 90 days after surgery (See table 5).

TABLE 5
Number of readmissions in the EASI-study (CABG-study). The number
of patients that had to be re-admitted to the hospital due
to clinical disease in the period post-surgery. Readmittance
was scored in the period of 28 days after operation, and in
the period ranging from 29-90 days after operation, and for
the total period of 90 days after operation. Readmittance was
reduced in patients receiving EA-230 treated group.

| | CABG-study | CABG-study | Total CABG- | |
|---|---|---|---|---|
| Table of Re- | Re-admission | Re-admission | study | Total |
| admissions | day 28 | day 90 | Re-admission | patients |
| Placebo | 5 | 5 | 10 | 89 |
| EA-230 | 2 | 2 | 4 | 91 |
| Total | 7 | 7 | 14 | 180 |

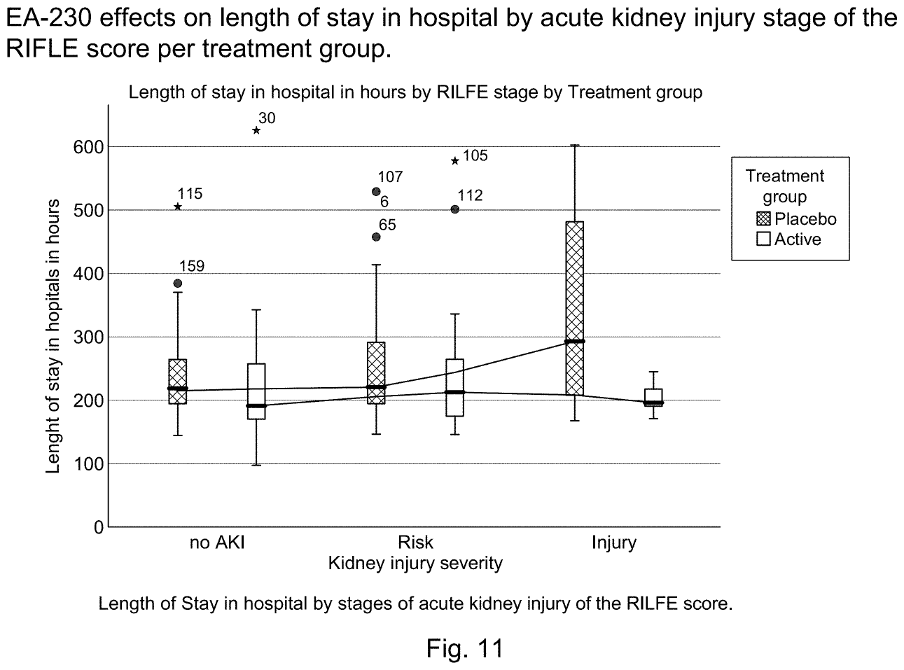

Furthermore, in the patient group treated with AQGV (SEQ ID NO:1), the number of patients suffering from AKI Injury was reduced, and when patients suffered AKI injury, these patients did not have a prolonged length-of-stay, as observed in the placebo group and length of stay was similar to patients having no AKI or patients being at risk of AKI (see FIG. 11).

Treatment with EA-230 herewith shows strong beneficial effects on recovery. EA 230-treated patients required significantly less hemodynamic therapy, regained post-surgical kidney function significantly faster and remained for a shorter period of time in the Intensive Care Unit (ICU) and in the hospital, as compared to placebo-treated patients.

These novel hemodynamic effects of EA-230 appear to be independent of anti-inflammatory effects of EA-230. In short, significant improvements of hemodynamic stability, kidney function and post-operative recovery of EA-230 treated patients relate to novel effects of EA-230 on blood vessel-permeability and blood vessel-contractility. EA-230 given during surgery shows significant improvements in patient recovery after surgery, over placebo patient. EA-230 treated patients are released faster from intensive care (p=0.0232) and hospital (p=0.0015). EA-230 improves hemodynamic stability (p=0.006) and kidney function (p=0.003). Whilst the primary endpoint—short term inflammatory cytokine (IL-6) reduction—was missed, long-term patient recovery was significantly improved by EA-230. Throughout surgery, EA-230 was shown to be safe and well tolerated. In conclusion, EA-230 given during surgery significantly improves recovery after surgery.

Significant improvement was found of hemodynamic stability (reducing fluid therapy and blood pressure medication; p=0.006), with: significant improvement of kidney function (improved glomerular filtration rate reduces plasma creatinine; p=0.003), significant reduction of patients suffering from adverse fluid retention during recovery (2 for EA-230, 9 for placebo; p=0.03), and considerable reduction of re-admissions to the hospital in the 90 days after surgery (4 for EA 230, 10 for placebo; p=0.09).

Further Analysis Biomarkers Related to Vasostriction and/or Vasodilation

In view of the effects observed on hemodynamics and kidney function, plasma samples are further analyzed with regard to selected biomarkers. Plasma samples of control patients and patients receiving the EA-230 are analyzed with regard to biomarkers Endothelin-1, VEGF, Angiotensine II and cAMP and natriuretic peptides. The following assays are used to determine the levels of the biomarkers

In Vitro Effects of EA-230 and AQGV Analogs

In an in vitro transwell assay the effects of the AQGV (SEQ ID NO:1) peptide (EA-230), and analogs thereof, is tested on human endothelial cells. Briefly, endothelial cells are cultured in transwell culture dishes and culture medium is supplemented with hemodynamic peptides, and analogs thereof, or control compounds known to affect endothelial layer permeability, vasoconstriction and/or vasodilation. Suitable human endothelial cells are e.g., HUVECs (Park et al., Stem Cell Rev. 2 (2): 93-102, 2006; Jiménez et al., Cytotechnology 65, 1-14, 2012) and HMEC-1 (Ades E W, et al. J. Invest. Dermatol. 99(6): 683-690, 1992). The permeability of the endothelial layer is determined by measuring the penetration of a macromolecule. Furthermore, levels of biomarkers are also determined in culture medium. Experiments are carried as outlined e.g., in Cox et al., Shock, 43(4):322-6; 2015. In HUVEC permeability tests, established human endothelial vascular cells (HUVEC), capable of lining blood vessels, are grown in cell-culture (i.e., n=5) on sieves, in multiple test formats, allowing determination of leak-through products depending on various test-concentrations of EA-230 peptide or placebo controls used, establishing pharmacological parameters of EA-230-peptide-effects on permeability in human cells.

Also, Bravo et al (J. Pharmacol. Toxicol. Methods 2018 January-February; 89:47-53) developed an impedance-based contraction assay using the xCELLigence RTCA MP system. This technology utilizes special 96-well E-plates with gold microelectrode arrays printed in individual wells to monitor cellular adhesion by recording the electrical impedance in real time. The impedance change (percentage vs. control) can be used as the readout for cellular contraction. Established human aortic smooth muscle cells (HaSMC), capable of contracting blood vessels, are grown in cell-culture (i.e., n=3) on gold-electrodes, in multiple test formats, allowing electrical-impedance-determination of endothelin-1 induced smooth muscle cell contractions, depending on various test-concentrations of EA-230-peptide or placebo controls used, establishing pharmacological parameters of EA-230-effects on contractility in human cells. In addition, isolated aneurysmatic (n=3)/control (n=3) patient human aortic smooth muscle cells (APaSMC), differentially capable of contracting blood vessels, are grown in cell-cultures on gold-electrodes in multiple test formats, allowing electrical-impedance-determination of ionomycin-induced smooth muscle cell contractions of patient-versus-control cells, depending on various test-concentrations of EA-230-peptide or placebo controls used, detecting effects of EA-230 in patient cells.

1.-48. (canceled)

49. A method of treating a subject in need of maintaining or improving hemodynamic stability, a reduction in adverse vascular permeability, and/or a reduction in fluid retention, the method comprising:
administering to the subject peptide(s) comprising at least 50% amino acids that are autophagy inhibiting amino acids,
wherein the autophagy inhibiting amino acids are selected from the group consisting of alanine, glutamine, glycine, valine, leucine, isoleucine, proline, and arginine.

50. The method according to claim 49, wherein the peptide(s) comprise(s) at least 75% of autophagy inhibiting amino acids.

51. The method according to claim 49, wherein the peptide(s) consist(s) of said autophagy inhibiting amino acids.

52. The method according to claim 49, wherein the peptide(s) comprise(s) at least 50% alanine, glutamine, and leucine.

53. The method according to claim 49, wherein the peptide(s) comprise(s) at most 30% amino acids selected from the group consisting of glycine, valine, isoleucine, proline, and arginine.

54. The method according to claim 49, wherein at least two different peptides are administered to the subject, each said peptide comprising at least 50% amino acids selected from the group consisting of alanine, glutamine, glycine, valine, leucine, isoleucine, proline, and arginine.

55. The method according to claim 49, wherein at least two peptides vary in length from 4 to 30 amino acids.

56. The method according to claim 49, wherein the subject has been subjected to severe trauma.

57. The method according to claim 49, wherein the subject has been subjected to cancer treatment.

58. The method according to claim 49, wherein the subject is suffering from capillary leakage syndrome.

59. The method according to claim 56, wherein the severe trauma is surgery.

60. The method according to claim 57, wherein the cancer treatment comprises treatment with an antineoplastic or immunomodulating agent.

61. The method according to claim 58, wherein the subject is suffering capillary leakage syndrome from an adverse drug reaction.

62. The method according to claim 49, wherein the subject has impaired kidney function.

63. The method according to claim 49, further comprising:
reducing use by the subject of a vasopressive agent.

64. The method according to claim 49, further comprising:
reducing the subject's fluid intake.

65. The method according to claim 49, wherein the peptide(s) comprise(s) at least 50% amino acids selected from the group consisting of alanine, glutamine, glycine, and valine.

66. The method according to claim 49, wherein the peptide(s) consist(s) of amino acids selected from the group consisting of alanine, glutamine, glycine, and valine.

67. The method according to claim 49, wherein at least one peptide is/are in the form of a salt of peptide-organic acid.

68. A pharmaceutical formulation comprising at least one peptide comprising at least 50% of autophagy inhibiting amino acids amino acids,
wherein an autophagy inhibiting amino acid is selected from the group consisting of alanine, glutamine, glycine, valine, leucine, isoleucine, proline, and arginine.

69. The pharmaceutical formulation of claim 68, wherein the at least one peptide comprises at least 75% of autophagy inhibiting amino acids.

70. The pharmaceutical formulation of claim 69, wherein the at least one peptide consists of 100% autophagy inhibiting amino acids.

71. The pharmaceutical formulation of claim 68, wherein the at least one peptide comprises at least 50% amino acids selected from the group consisting of alanine, glutamine, and leucine.

72. The pharmaceutical formulation of claim 68, wherein the at least one peptide comprises at most 30% amino acids selected from the group consisting of glycine, valine, isoleucine, proline, and arginine.

73. The pharmaceutical formulation of claim 68, comprising at least two different peptides each comprising at least 50% amino acids selected from the group consisting of alanine, glutamine, glycine, valine, leucine, isoleucine, proline, and arginine.

74. The pharmaceutical formulation of claim 68, wherein the peptides vary in length from 4 to 30 amino acids.

75. The pharmaceutical formulation of claim 68, comprising at least 0.85 mol/L of the peptide(s).

76. The pharmaceutical formulation of claim 68, together with at least one pharmaceutically acceptable excipient.

77. The pharmaceutical formulation of claim 68, wherein the peptide(s) comprise(s) at least 50% amino acids selected from the group consisting of alanine, glutamine, glycine, and valine.

78. The pharmaceutical formulation of claim 77, wherein the peptide(s) consist(s) of amino acids selected from the group consisting of autophagy inhibiting amino acids alanine, glutamine, glycine, and valine.

79. The pharmaceutical formulation of claim 78, wherein the peptide(s) is a salt selected from the group consisting of peptide-acetate, peptide-tartrate or peptide-citrate.

80. A method of treating a subject suffering from Clarkson's disease (CLS), the method comprising:
administering the pharmaceutical formulation of claim 68 to the subject so as to treat the CLS.
